SEQUENCE LISTING (23 sequences)

SEQ ID NO: 1 - DNA, 20 bp, artificial sequence
  Primer
gaaagaagatttcaccaggg

SEQ ID NO: 2 - DNA, 20 bp, artificial sequence
  Primer
ccttcaggtaatgcagaatc

SEQ ID NO: 3 - DNA, 20 bp, artificial sequence
  Primer
tcttttgcgtcgccagccga

SEQ ID NO: 4 - DNA, 20 bp, artificial sequence
  Primer
accaggcgcccaatacgacc

SEQ ID NO: 5 - DNA, 20 bp, artificial sequence
  Primer
aaatgggaaatgtgatccag

SEQ ID NO: 6 - DNA, 21 bp, artificial sequence
  Primer
agtatacctgtctaagaccac

SEQ ID NO: 7 - DNA, 22 bp, artificial sequence
  Primer
cgggtagatgaaattatttggg

SEQ ID NO: 8 - DNA, 20 bp, artificial sequence
  Primer
gatttcctttgacaccaacc

SEQ ID NO: 9 - DNA, 20 bp, artificial sequence
  Primer
actagaaatgcaacaggatg

SEQ ID NO: 10 - DNA, 20 bp, artificial sequence
  Primer
gatttaagctctgtgttccc

SEQ ID NO: 11 - DNA, 20 bp, artificial sequence
  Primer
cggcgaaggctacacttacg

SEQ ID NO: 12 - DNA, 21 bp, artificial sequence
  Primer
ccaccagcttttggatcacca

SEQ ID NO: 13 - DNA, 20 bp, artificial sequence
  Primer
ccagagaacaaaaggacaag

SEQ ID NO: 14 - DNA, 21 bp, artificial sequence
  Primer
tttgactgtccagtaaggtag

SEQ ID NO: 15 - DNA, 20 bp, artificial sequence
  Primer
ccaagaggaagaagaaagtg

SEQ ID NO: 16 - DNA, 22 bp, artificial sequence
  Primer
agtcctaattctcttgaaggtc

SEQ ID NO: 17 - DNA, 21 bp, artificial sequence
  Primer
atcagagtaacagctatccac

SEQ ID NO: 18 - DNA, 20 bp, artificial sequence
  Primer
cccaggttgactaaatacac

SEQ ID NO: 19 - DNA, 20 bp, artificial sequence
  Primer
aagagagctagaggcatatc

SEQ ID NO: 20 - DNA, 20 bp, artificial sequence
  Primer
atgcttcttttctgaacctg

SEQ ID NO: 21 - DNA, 20 bp, artificial sequence
  Primer
cccaccgtgttcttcgacat

SEQ ID NO: 22 - DNA, 21 bp, artificial sequence
  Primer
tctttgggaccttgtctgcaa

SEQ ID NO: 23 - RNA, 21 nt, artificial sequence
  shRNA cDNA
cugcaaguauguucacuauga

(19) United States
(12) Patent Application Publication
(10) Pub. No.: US 2021/0164992 A1
(43) Pub. Date: Jun. 3, 2021

(54) COMPOSITIONS AND METHODS RELATED TO INNATE RESPONSE TO DNA AND REGULATION OF INTERFERON-BETA

(71) Applicant: New York University, New York, NY (US)

(72) Inventor: Ian MOHR, New York, NY (US)

(21) Appl. No.: 17/262,692

(22) PCT Filed: Jul. 22, 2019
(86) PCT No.: PCT/US2019/042784

§ 371 Date: Jan. 22, 2021

Related U.S. Application Data
(60) Provisional application No. 62/702,009, filed on Jul. 23, 2018.

(51) Int. Cl. G01N 33/68; A61K 31/192

(52) U.S. Cl. CPC G01N 33/6866; G01N 2333/565; A61K 31/192

(57) ABSTRACT

Provided are methods of modulating the type I interferon (IFN) pathway in an individual. The methods involve administering to an individual one or more agents that inhibit expression or function of METTL3, or METTL14, or ALKBH5, thereby modulating the type I IFN pathway. The agent that inhibits the expression and/or function ALKBH5 results in decreased interferon β cytokine production and/or decreased IFNB1 mRNA, and is useful for treating autoimmune and inflammatory conditions. The agent that inhibits expression or function of METTL3 and/or METTL14 result in increased IPNβ cytokine production and/or increased IFNB 1 mRNA, and is useful for treating infections and cancer. Methods for screening and identifying enzyme inhibitors are also provided.

### CROSS REFERENCE TO RELATED APPLICATION

This application claims priority to U.S. provisional patent application No. 62/702,009, filed Jul. 23, 2018, the disclosure of which is incorporated herein by reference.

### STATEMENT REGARDING FEDERALLY SPONSORED RESEARCH OR DEVELOPMENT

This invention was made with government support under grant nos. GM056927 and AI073898 awarded by the National Institutes of Health. The government has certain rights in the invention

### FIELD

This disclosure relates generally to compositions and methods pertaining to modulating the Type I interferon signaling pathway, and therapeutic approaches based on such modulation for conditions such as autoimmune disorders, inflammatory conditions, infections, and cancer.

### BACKGROUND

Differential chemical modification of mRNA in theory provides a powerful means to dynamically alter gene expression. In particular, methylation of adenosine at the N⁶ position (m⁶A) constitutes the most widespread internal base modification to mRNA (Yue et al., 2016; Roundtree et al., 2017). A methyltransferase writer core complex composed of the METTL3 catalytic subunit, METTL14, and WTAP installs m⁶A marks on nascent mRNA cotranscriptionally (Lu et al., 2014). Distributed primarily within mRNA coding sequences and reportedly enriched at the start of 3′-exons and proximal to termination codons, m⁶A is also found within 3′-untranslated regions (UTRs) and extended 5′-cap structures where the 2′-O-methyl nucleotide immediately adjacent to the 7-methylguanosine (m⁷G) cap is an adenosine (Dominissini et al., 2012; Meyer et al., 2012). In addition to modifying RNA secondary structure (Roost et al., 2015; Liu et al, 2015; 2017), m⁶A is specifically recognized by a family of “reader” proteins, including YTH-domain containing proteins that reside primarily in the cytoplasm (YTHDF1, YTHDF2) or the nucleus (YTHDC1, YTHDF3) (Patil et al., 2018). Identification of demethylases FTO and ALKBH5 capable of erasing m⁶A marks in vitro suggested that m⁶A modifications were potentially dynamic and reversible, sculpted by the opposing actions of writers that install m⁶A and erasers that remove it (Jia et al, 2011; Zheng et al, 2015). Recently, however, FTO was shown to have limited capacity to demethylate internal m⁶A residues with its preferred substrate being N⁶, 2′-O-dimethyl-adenosine (m⁶Aₘ) located beside the m⁷G cap (Mauer et al, 2017). Although m⁶A methylation occurs to nascent pre-mRNA, it is predominately stable in the cytoplasm until the mRNA decays (Ke et al, 2017). While this argues against methylation removal in the cytoplasm, internal m⁶A removal by a demethylase like ALKBH5 within the nucleus could not be excluded and remains possible (Ke et al, 2017). Numerous aspects of RNA biology and metabolism have been reportedly regulated by m⁶A mRNA modification including nuclear processing and export (Zheng et al, 2015; Fustin et al., 2013; Zheng et al., 2017; Haussman et al, 2016), translation (Meyer et al., 2015; Coots et al., 2017; Zhou et al., 2018) and mRNA decay (Wang et al, 2014; Du et al., 2016; Chen & Shyu, 2017; Edupuganti et al., 2017). In addition, mRNA modification by m⁶A influences development, differentiation and reprogramming, circadian rhythm, cell cycle, disease pathogenesis and stress responses (Fustin et al., 2013; Aguilo et al., 2015; Mer et al., 2015; Zhou et al., 2015; Wojtas et al, 2017; Vu et al., 2017; Wen et al., 2018).

Virus-encoded mRNAs are also chemically modified by m⁶A, and a role for m⁶A in infection biology is emerging. While m⁶A has been detected in viral RNA, precisely how host enzymes that control m⁶A modification levels impact virus reproduction remains unclear. For two RNA viruses that replicate in the nucleus, cellular m⁶A functions stimulate virus reproduction. The METTL3/14 m⁶A writer complex reportedly promotes HIV replication whereas m⁶A erasers ALKBH5 and FTO suppress it (Lichinchi et al., 2016a; Kennedy et al, 2016; Tirumuru et al, 2016). However, different mechanisms have been proposed to account for this including differential nuclear export, translation, and virus genome replication. Similarly, the METTL3 methyltransferase promotes influenza A virus replication and m⁶A sites in the viral HA segment are required for proper protein expression and pathogenicity in mice (Courtney et al., 2017). Recently, RNA viruses that replicate exclusively in the cytoplasm have been found to contain m⁶A in their (+) sense RNA genomes. In contrast with nuclear RNA viruses, m⁶A writers METTL3/14 limited HCV infectious particle production and protein expression without detectably changing RNA replication (Gokhale et al., 2016; Gonzales-van Horn & Sammy, 2017). The FTO demethylase, however, stimulated HCV protein expression. In addition, METTL3/14 restricted Zika virus reproduction whereas FTO and ALKBH5 demethylases enhanced it. Zika virus infection also enriched for m⁶A modifications of host mRNAs but did not detectably change the abundance of host anti-viral transcripts (Lichinchi et al., 2016b). DNA viruses such as SV40, Adenovirus and herpesviruses replicate in the nucleus and produce mRNAs that contain m⁶A (Lavi & Shatkin, 1975; Sommer et al., 1976; Moss et al., 1977; Tan et al., 2018; Tsai et al, 2018; Hesser et al., 2018). Intriguingly, interfering with the host m⁶A machinery in cells infected with the gamma herpesvirus KSHV resulted in complex pro- and anti-viral outcomes that were dependent on the cell type analyzed (Hesser et al., 2018). How cellular functions that control steady-state mRNA m⁶A levels impact the reproduction of large DNA viruses and the DNA-sensing pathways that impact host innate defenses remains unknown.

Infection with human cytomegalovirus (HCMV), a large DNA virus that replicates within the nucleus, is predominately asymptomatic in healthy individuals (Britt, 2008; Boeckh & Geballe, 2011). However, HCMV causes life-threatening disease among the immunocompromised, including solid-organ or stem cell transplant recipients (Ljungman et al., 2010; Razonable & Humar, 2013), and is a significant source of congenital morbidity and mortality among newborn infants in the developed world (Cannon et al, 2010; Manicklal et al., 2013). Unlike many viruses, including alpha and gamma herpesvirus subfamily members, the beta herpesvirus HCMV does not globally impair ongoing host protein synthesis. Instead, extensive remodeling of the host translational landscape translation plays a critical role in regulating HCMV productive replication (McKinney et al., 2014; Tlrosh et al., 2015). Significantly, changes in host gene expression induced in response to HCMV can either stimulate or restrict acute virus reproduction. While some HCMV-induced alterations to host gene expression require de novo expression of viral genes, others do not and are in fact triggered by exposure to the viral dsDNA genome. Whether any of these responses to HCMV are impacted by cellular m⁶A RNA modification enzymes and how this might influence HCMV reproduction is unknown. Furthermore, the unanticipated extent to which HCMV manipulates host gene expression post-transcriptionally, balancing expression of proviral cellular factors and anti-viral host responses, make it a powerful model to investigate how the cellular m⁶A modification machinery impacts virus reproduction. There is accordingly an ongoing and unmet need to determine how cellular m⁶A writers, erasers, and reader proteins interact with cytoplasmic dsDNA, and to exploit these mechanisms for treating a variety of conditions that are influenced by their functions. The present disclosure is pertinent to these needs.

### BRIEF SUMMARY

The present disclosure relates to methods for treating a variety of disorders that involve pathological deregulation of inflammatory cytokine production. The methods involve modulating the Type I interferon (IFN) signaling pathway, such as by increasing or decreasing expression of, or inhibiting the function of, enzymes known in the art as METTL3, METTL14, and ALKBH5.

In embodiments, inhibiting the expression and/or function of METTL3, METTL14, or a combination thereof, increases production of interferon beta 1 (IFNB1) mRNA, which encodes the cytokine IFNβ, and IFNβ cytokine production. In contrast, inhibiting the expression and/or function of ALKBH5 reduces expression of IFNB1 mRNA and IFNβ cytokine production. Thus, by manipulating IFNB1 mRNA and IFNβ cytokine production, the disclosure provides approaches for addressing distinct disorders, some of which may benefit from increasing inflammatory cytokine production (such as various types of cancers and microbial/viral infections) or decreasing inflammatory cytokine production (such as for autoimmune diseases, and acute and chronic inflammation, and related conditions).

Embodiments of the disclosure are shown in non-limiting examples which demonstrate that type I IFN production triggered by double-strand (ds) DNA or human cytomegalovirus (HCMV) is controlled by the cellular METTL3/METTL14 m⁶A-methyltrasferase and ALKBH5 demethylase. While METTL14-depletion reduced virus reproduction and stimulated dsDNA or HCMV-induced IFNB1 mRNA accumulation, ALKBH5-depletion had the opposite effect. Depleting METTL14 increased both nascent IFNB1 mRNA production and stability in response to dsDNA. By contrast, ALKBH5-depletion reduced nascent IFNB1 mRNA production without detectably influencing IFNB1 mRNA decay. Genome-wide transcriptome profiling following ALKBH5-depletion identified differentially-expressed genes regulating anti-viral immune responses, while METTL14-depletion altered pathways impacting metabolic reprogramming, stress responses, and aging. It is shown in this disclosure that IFNB1 mRNA was m⁶A-modified within both the coding sequence and the 3′-UTR. The disclosure thus establishes that the host m⁶A modification machinery controls IFNB1 production triggered by HCMV or dsDNA. Moreover, the disclosure demonstrates in non-limiting embodiments that responses to non-microbial dsDNA in uninfected cells, which shape host immunity and contribute to autoimmune disease, are regulated by enzymes controlling m⁶A-epitranscriptomic changes. Accordingly, data presented herein support inhibition of the expression and/or functions of METTL14, METTL3, and ALKBH5 as an approach to treating conditions related to inflammatory cytokine production.

In one approach, the disclosure provides a method of modulating the type I IFN pathway in an individual by administering to an individual in need thereof one or more agents that inhibit expression or function of METTL3, or METTL14, or ALKBH5, thereby modulating the type I IFN pathway.

In an embodiment, the method comprises administering to an individual an agent that inhibits the expression and/or function of ALKBH5. This results in decreased IFNβ cytokine production and/or decreased IFNB1 mRNA in cells of the individual. The decrease may be relative to a control IFNβ cytokine production or IFNB1 mRNA value, respectively, from cells to which the agent has not been administered. In embodiments, the pertinent cells comprise any potential type I interferon producing cells, examples of which include but are not limited to fibroblasts, epithelial cells, dendritic cells, macrophage, monocytes, stromal cells, and parenchymal cells.

Targeting expression and/or function of ALKBH5 may be used for any individual that has an autoimmune disease or inflammatory disease. This approach is expected to reduce the severity of the autoimmune disease or the inflammatory disease. In certain non-limiting embodiments, the autoimmune diseases and/or inflammatory disease may be positively correlated with the presence of cytoplasmic doubled stranded (dsDNA) in cells of the individual. The cytoplasmic dsDNA, in the case of an autoimmune disease, may be self-dsDNA, and thus may have an antigenic function. In embodiments, the presence of cytoplasmic DNA may be detected, but it is not required. In embodiments, methods of the disclosure are used to treat conditions that are related to pathological deregulation of inflammatory cytokine production, regardless of the presence or absence of cytoplasmic dsDNA.

In an embodiment, a method of the disclosure comprises administering to an individual one or more agents that inhibit the expression or function of METTL3, or METTL14, or both. This results in increased IFNβ cytokine production and/or increased IFNB1 mRNA in cells of the individual. This increase may be relative to a control IFNβ cytokine production or IFNB1 mRNA value, respectively, from cells to which the agent has not been administered. In embodiments, this approach may be used for individuals who have been diagnosed with or are suspected of having cancer, or an infection caused by an intracellular parasite. The intracellular parasite may be a DNA virus, or an infectious microbe, including but not necessarily limited to intracellular pathogenic bacteria, pathogenic eukaryotic microbes, and pathogenic fungi. In non-limiting embodiments, the cancer or the infection may be correlated with the presence of cytoplasmic dsDNA in cells of the individual.

In another approach, the disclosure provides a method for identifying whether or a test agent affects the function of METTL3, METTL14 or ALKBH5. This comprises:

i) providing a plurality of eukaryotic cells that can produce IFNB1 mRNA and/or IFNβ cytokine;

ii) introducing one or more test agents into the cells; and

iii) determining whether or not there is a change in the amount of IFNβ cytokine production and/or IFNB1 mRNA in the cells subsequent to ii).

Detecting the change indicates the test agent may inhibit the function of METTL3/14 and/or ALKBH5, depending on whether there is an increase or decrease in IFNβ cytokine production and/or IFNB1 mRNA, as those changes are described above. For example, a decrease in the amount of IFNβ cytokine production and/or IFNB1 mRNA indicates the agent may inhibit the function of the ALKBH5. In order to provide additional evidence of the ALKBH5 inhibition function of the test agent, the disclosure optionally includes testing the agent in control cells with inhibited or reduced ALKBH5 expression, wherein a lack of a decrease in the amount of IFNβ cytokine production and/or IFNB1 mRNA by the control cells indicates the agent inhibits the function of the ALKBH5. This same strategy can be implemented for inhibitors of METTL3 and METTL14, provided that the testing is designed to detect an increase in IFNβ cytokine production and/or an increase in IFNB1 mRNA.

### BRIEF DESCRIPTION OF THE FIGURES

FIG. 1. Accumulation of the cellular m⁶A machinery is induced by HCMV. A) Regulation of m⁶A levels in mRNA by the opposing action of a multi-subunit methyltransferase and demethylases. A subset of reader proteins that recognize m⁶A-modified residues are depicted. B) NHDFs were mock-infected (uninfected) or infected with HCMV (MOI=3). At the indicated hours post-infection (hpi), total protein was collected, fractionated by SDS-PAGE and analyzed by immunoblotting with the indicated antibodies. Actin represents a host antigen whose levels remain unchanged during infection. C) As in B except cells were infected with UV-inactivated HCMV and GAPDH was used as a control host antigen. D) Total RNA collected from HCMV-infected cells (MOI=3) at the indicated times (h) post-infection. For each of the indicated genes, mRNA was analyzed by real-time qPCR and normalized to PPIA mRNA levels. Three biological replicates were performed. Error bars indicate SEM. E) Uninfected NHDFs or NHDFs infected with HCMV (MOI=3) were treated with the mTOR-active site inhibitor PP242 or DMSO. At the indicated times (h post-infection, h.p.i.), total protein was collected, fractionated by SDS-PAGE, and analyzed by immunoblotting with the indicated antisera. GAPDH served as a loading control. The change in 4E-BP1 migration in uninfected cells from a slow migrating, hyperphosphorylated species (hyper) to a faster migrating hypophosphorylated (hypo) form validates the activity of PP242.

FIG. 2. Control of HCMV replication by the host RNA m⁶A METTL3/14 methyltransferase and ALKBH5 demethylase. A) NHDFs were treated with control, non-silencing siRNA, siRNA specific for the m⁶A methyltransferase METTL3 or METTL14 subunits, or siRNA specific for the ALKBH5 demethylase. After 72 h, cultures were infected with HCMV (MOI=0.05) and the infection allowed to proceed for 7 d. Supernatants were harvested and virus titer (TCID/50) determined using NHDFs. Error bars indicate SEM. **p<0.01; ****p<0.0001 by student's t-test. B) As in A except total protein was harvested, fractionated by SDS-PAGE and the accumulation of viral proteins encoded by representative immediate-early (1E1/2), early (UL44), or late (pp28) genes measured by immunoblotting with the indicated antisera. GAPDH serves as a loading control. C) NHDFs treated with control, non-silencing siRNA or siRNA specific for METTL3, METTL14 or ALKBH5 were infected with HCMV (MOI=3). After 6 h, total RNA was harvested and interferon beta mRNA levels quantified by qRT-PCR. Three biological replicates (n=3) were performed and error bars represent SEM. *P<0.05, **P<0.005 by Student's t-test. D) NHDFs were treated with control, non-silencing siRNA, or siRNA specific for METTL14. After 72 h, cultures were either treated with 1 μM JAK inhibitor (Pyridone 6) or DMSO and infected with HCMV (MOI=0.05). Supernatants were harvested and virus titer (TCID/50) determined after 7 d using NHDFs.

FIG. 3. Induction of type I interferon in response to dsDNA is regulated by RNA m⁶A METTL3/14 methyltransferase and ALKBH5 demethylase. A) NHDFs treated with control, non-silencing siRNA, or siRNAs specific for METTL14 or ALKBH5 were incubated with H₂O or dsDNA. After 9 h, total RNA was collected and IFNB1 mRNA measured by qRT-PCR. Error bars indicate SEM. *p<0.05; **p<0.01 by student's t-test. B) RNA isolated from NHDFs treated with dsDNA for 6 h was immunoprecipitated with anti-m⁶A antibody. RNA enriched in the immune complex was analyzed by qRT-PCR using primers specific for the indicated genes. C) Two regions of the IFNB1 transcript are enriched for m⁶A peaks. Visualization of m⁶A-Seq results shows regions of enrichment for m⁶A-IP (red) over input (blue) for three biological replicates of NHDFs transfected with dsDNA for 12 h. Specific regions enriched for m⁶A-modifications were identified using ExomePeak (see Supplemental Methods) and are shown as horizontal black lines. m⁶A DRACH motifs (Linder et al., 2015) are shown as red boxes. No IFNB1-mapping reads were detected in control (no dsDNA) IP or input datasets (data not shown), consistent with the minimal, background presence of IFNB1 transcripts in untreated cells. The transcript structure of IFNB1 is denoted in dark blue (3′ UTR & 5′ UTR) and blue (CDS). D) NHDFs treated as in A were incubated with H₂O or dsDNA. After 24 h supernatants or known concentrations of IFNβ standards were placed on indicator cells and the amount of IFNβ activity quantified. Error bars indicate SEM. *p<0.05; ***p<0.001 by student's t-test. E) NHDFs treated with control, non-silencing siRNA or the indicated siRNAs specific for the demethylase ALKBH5 and/or m⁶A readers YTHDF1, YTHDF2, YTHDF3 were exposed to dsDNA. After 9 h, total RNA was collected and IFNB1 mRNA quantified by qRT-PCR. Error bars indicate SEM. *p<0.05; **p<0.01 by student's t-test.

FIG. 4. Control of genome-wide responses to dsDNA by m⁶A demethylase ALKBH5 and m⁶A methylase subunit METTL14. Volcano plots show differentially expressed genes (adjusted p value <0.01) identified from RNA-Seq of polyadenylated RNA, collected from cells transfected with (A) ALKBH5 siRNA [siALKBH5 (n=3 biological replicates)], or (B) METTL14 siRNA [siMETTL14 (n=3 biological replicates)], and stimulated with dsDNA for 12 h. Genes upregulated versus a non-silencing siRNA control (stimulated with dsDNA for 12 h, n=3 biological replicates) are shown in red, while downregulated genes are shown in blue. Non-regulated genes are shown in grey. (C) 1,447 significantly regulated genes (p<0.01) overlap between datasets generated using siALKBH5 and siMETTL14. A scatter plot of these shows genes that are commonly up- or down-regulated following either METTL14 or ALKBH5 silencing highlighted in red and blue, respectively. Genes regulated in a reciprocal manner are highlighted in green (upregulated when METTL14 is silenced, downregulated when ALKBH5 is silenced) and yellow (downregulated when METTL14 is silenced, upregulated when ALKBH5 is silenced. The red circle highlights the IFNB1 mRNA. (D) Heatmap depicting 349 interferon-stimulated genes (ISGs), colored according to log 2fold change in expression versus the non-silencing siRNA control. (E-F) Pathway analyses (GO direct terms) of significantly differentially expressed genes from (A) and (B) were conducted using DAVID and filtered according to a Benjamini-Hochberg procedure (<0.05).

FIG. 5. Control of IFNB1 mRNA biogenesis and decay by ALKBH5 and METTL14. NHDFs were treated with control, non-silencing siRNA or siRNA specific for the METTL14 m⁶A methyltransferase subunit or the ALKBH5 demethylase for 72 h. A) NHDFs treated with the indicated siRNAs were transfected with dsDNA in the presence of DMSO or the JAK inhibitor (Pyridone 6). At the indicated times total RNA was harvested and the abundance of IFNB1 mRNA quantified by qRT-PCR. Error bars indicate SEM. *p<0.05; **p<0.01; ***p<0.001 by student's t-test. The bars are from left to right for each time point are Control, METTL14, and ALKBH5. B) As in A except cultures were pulse-labeled for 30 min. with 5-ethyluridine (EU) at either 7 h or 10 h post-exposure to dsDNA. Immediately following the EU pulse, nuclear RNA was collected, nascent EU-containing RNA isolated and the abundance of IFNB1 mRNA quantified by qRT-PCR. Error bars indicate SEM. *p<0.05; **p<0.01 by student's t-test. C) Left panel: NHDFs treated with the indicated siRNAs were exposed to dsDNA for 3 h and pulse-labeled for 2 h with 5-ethyluridine (EU). Free EU was washed out and total RNA harvested at the indicated time points. Following isolation of nascent EU-containing RNA, overall IFNB1 mRNA levels were measured by qRT-PCR and normalized to GAPDH. Error bars indicate SEM. *p<0.04 by student's t-test. Right Panel: As in left panel, except cultures were also treated with the JAK inhibitor (Pyridone 6). D) Model illustrating the relationship between IFNB1 mRNA levels, induced in response to dsDNA in uninfected cells or HCMV infection, and m⁶A levels in mRNA. The balance between methyltransferase METTL14 and demethylase ALKBH5 activities is depicted as a scale. METTL14 depletion (siMETTL14) tips the scale in favor of the ALKBH5 demethylase, which increases IFNB1 mRNA levels and is predicted to reduce m⁶A levels. In contrast, ALKBH5 depletion (siALKBH5) disrupts the balance in favor of methyltransferase activity which decreases IFNB1 mRNA and is predicted to increase m⁶A levels.

FIG. 6. mRNA abundance and ribosome footprints of m⁶A readers, writers, and erasers. (A) Abundance of selected mRNAs, relative to mock treatment, as profiled during a 72 hr HCMV infection. (B) Ribosome footprint profiling of the same mRNAs. All data were extracted from Tirosh et al., 2015.

FIG. 7. Cellular m⁶A machinery responds to HCMV infection and regulates virus gene expression. A) NHDFs were mock-infected (uninfected) or infected with HCMV (MOI=0.05). At the indicated hours post-infection (hpi), total protein was collected, fractionated by SDS-PAGE and analyzed by immunoblotting with the indicated antibodies. GAPDH represents a host antigen whose levels remain unchanged during infection. B) NHDFs were treated with control, non-silencing siRNA, siRNA specific for the m⁶A methyltransferase METTL14 subunit, or siRNA specific for the ALKBH5 demethylase. After 72 h, cultures were infected with HCMV (MOI=3). At the indicated h post-infection, total protein was isolated, fractionated by SDS-PAGE and analyzed by immunoblotting with the indicated antisera.

FIG. 8. Interfering with the host METTL3 m⁶A methylase and ALKBH5 demethylase selectively impacts HCMV, but not HSV1 or Vaccinia productive growth. NHDFs were treated with control, non-silencing siRNA, siRNA specific for the m⁶A methyltransferase METTL3 subunits, or siRNA specific for the ALKBH5 or FTO demethylase. After 72 h, cultures were infected with HCMV (MOI=0.05), HSV-1 (MOI=5×10⁻⁴), or Vaccinia virus (MOI=5×10⁻⁴). Supernatants were harvested after 7 d for HCMV, 72 h for HSV-1 and 48 h for Vaccinia virus and virus titer (TCID/50) determined using NHDFs for HCMV or by plaque assay in Vero cells for HSV-1 and Vaccinia. The error bars indicate SEM. **, P≤0.01; ***, P≤0.001, by Student's t test

FIG. 9. METTL14 or ALKBH5-depletion does not detectably impact STAT1 phosphorylation in response to IFNb. NHDFs transfected with control, non-silencing siRNA or siRNA specific for METTL14 or ALKBH5 were untreated or treated with 100 U IFNb. After 4 h, total protein was collected, fractionated by SDS-PAGE and analyzed by immunoblotting using the indicated anti-sera.

FIG. 10. Assessment of sequencing quality. (A) heatmap of sample-to-sample distances comparing all twenty-seven samples sequenced in this disclosure. Sample siALKBH5-12 hrs was excluded from further analyses due to aberrant clustering. (B) principal component analysis (PCA) plot showing the same dataset organized according the principal components 1 and 2.

FIG. 11. Control of genome-wide responses to dsDNA by m⁶A demethylase ALKBH5 and m⁶A methylase subunit METTL14. Volcano plots show differentially expressed genes (adjusted p value <0.01) identified from RNA-Seq of polyadenylated RNA, collected from cells transfected with (A) siALKBH5 (n=3 biological replicates), or (B) siMETTL14 (n=2 biological replicates), and stimulated with dsDNA for 6 h. Genes upregulated versus an non-silencing siRNA control (stimulated with dsDNA for 6 h, n=3 biological replicates) are shown in red, while downregulated genes are shown in blue. Non-regulated genes are shown in grey. (C) Heatmap depicting 349 interferon-stimulated genes (ISGs), colored according to log 2fold change in expression versus the non-silencing siRNA control. (D-E) Pathway analyses (GO direct terms) of significantly differentially expressed genes from (a) and (b) were conducted using DAVID and filtered according to a Benjamini-Hochberg procedure (<0.05).

FIG. 12. Profiling of regulated interferons and siRNA targets Following stimulation with dsDNA, increased cellular transcription of A, IFNB1, B, IL-6, and C, IFNL1 is variably modulated by siRNA-mediated suppression of METTL14 (orange) and ALKBH5 (purple) relative to siRNA controls at 6 and 12 hrs post-stimulation. Statistically significant differences log 2 fold changes (adjusted p value <0.05) are highlighted by asterisks (*). Error bars represent the log 2 standard error. D, median normalized transcripts counts (mRNA abundance) for IFNB1, IFNL1, and IL6. Error bars indicated the standard error. E, heatmap showing relative expression of the three regulated interferons and two siRNA targets. F, heatmap showing relative expression of genes upregulated by METTL14-depletion and downregulated by ALKBH5-depletion.

FIG. 13. Pathway analysis of differentially expressed genes in siALKBH5-treated cells A) after a 6 or 12 (B) hrs post dsDNA stimulation. Enriched pathways (GO direct terms) were identified via David (REF) using all significantly upregulated (red—indicated by brackets) or downregulated (blue) gene sets. Only pathways with a Benjamini-Hochberg score <0.05 are shown.

FIGS. 14A & 14B. Pathway analysis of differentially expressed genes in siMETTL14-treated cells. A. Enriched pathways (GO direct terms) 6 hours post-DNA stimulation were identified via David (REF) using all significantly upregulated (red) or downregulated (blue) gene sets. Only pathways with a Benjamini-Hochberg score <0.05 are shown. B. As in A except after 12 hours post-DNA stimulation

FIG. 15. Response of m⁶A machinery to dsDNA in uninfected cells. A) NHDFs exposed to buffer or dsDNA for 7 h were fixed, permeablized and stained with DAPI. Subcellular distribution of host m⁶A methylase subunits (METTL3, METTL14, WTAP), the demethylase ALKBH5, or m⁶A readers (YTHDF2, YTHDF3) was visualized by indirect immunofluorescence using the indicated antibodies. B) Total protein isolated from NHDFs exposed to buffer or transfected with dsDNA for the indicated times (h.p.t.=h post-transfection) was fractionated by SDS-PAGE and analyzed by immunoblotting with the indicated antisera. Actin is a loading control; MIDAS is a control showing dsDNA-induced protein accumulation. C. NHDFs were treated with control, non-silencing siRNA, or siRNA specific for the indicted targets (METTL14, ALKBH5, STING). After 72 h, cultures were treated with buffer or dsDNA for 6 h. Total protein was harvested, fractionated by SDS-PAGE and analyzed by immunoblotting with the antibodies indicated on the left. Actin is a loading control. D) As in C except cultures were fixed, permeabilized, and stained with DAPI. Subcellular IRF3 distribution was determined by indirect immunofluorescence using an IRF3-specific antibody.

FIG. 16. Graphs showing small molecule-based inhibition of ALKBH5. To obtain the data shown in the graphs, primary human fibroblasts were transfected with or without dsDNA. After 9 h, IFNB1 mRNA accumulation was measured by RT-PCR. Cultures were treated with vehicle (DMSO, 0 mM Rhein) or the indicated concentrations of Rhein either 24 h prior to transfection (−24 h) or at the time of transfection (0 h).

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The disclosure includes all polynucleotide sequences described herein, their complementary sequences, DNA equivalents of RNA and vice versa, all polynucleotide sequences that encode any protein described herein, and all amino acid sequences referred to herein.

Any polynucleotide and amino acid sequences that are described by way to a database entry are incorporated herein as they exist in the database on the effective filing date of this application or patent.

The present disclosure relates in part to cellular functions required for m⁶A RNA modification in response to the intracellular sensing or detection of dsDNA, as described further below.

In embodiments, the disclosure comprises modulating expression and/or function of one or more enzymes described herein. In an embodiment, the disclosure comprises modulating expression and/or function of ALKBH5 demethylase. In certain embodiments, expression and/or function of ALKBH5 demethylase is inhibited. Inhibition of ALKBH5 may be selective or specific. In embodiments, ALKBH5 is inhibited concurrently with one or both of YTHDF1 and YTHDF2.

In embodiments, the disclosure comprises modulating expression and/or function of m⁶A methylase subunit METTL3, or METTL14, or both METTL3 AND METTL14. In embodiments, expression and/or function of METTL14 is modulated. Inhibition of these enzymes may also be selective or specific. Thus, specifically or selectively modulating expression of any subunit of m⁶A methyltransferase is encompassed by this disclosure.

While aspects of this disclosure are illustrated using cells that comprise cytoplasmic dsDNA, determination that dsDNA is present is an optional aspect of the disclosure. Further, the disclosure includes treating disorders that are not necessarily positively correlated with the presence of cytoplasmic dsDNA. Nevertheless, if desired, the disclosure includes treating conditions that are associated with cytoplasmic DNA responses, including but not necessarily limited to self-responses to cytoplasmic dsDNA, including but not necessarily limited to cytoplasmic DNA that is involved in cytoplasmic dsDNA signaling. If desired, the presence of cytoplasmic dsDNA can be determined by from a biological sample obtained from an individual, or may be inferred based on a diagnosis of, for example, certain microbial and viral infections, and/or autoimmune disorders, inflammatory diseases, or cancer. Further, cytoplasmic dsDNA may originate from mitochondria, and it is also known that mitochondrial stress itself may trigger a type I interferon response. Addressing conditions related to type I interferon responses that are induced by mitochondrial DNA are therefore encompassed by this disclosure. Cytoplasmic dsDNA may also originate from the nucleus as cells age and undergo senescence.

The amino acid sequence of, and nucleotide sequence encoding, human ALKBH5 are known (NCBI Reference Sequence: NM_017758.4). The amino acid sequence of, and nucleotide sequence encoding, human METTL3 are known (NCBI Reference Sequence: NM_019852.5). Likewise, the amino acid sequence of, and the nucleotide sequence encoding, human METTL14 are known (NCBI Reference Sequence: NM_020961.4). The disclosure includes all of these sequences, all and splice variants and isoforms of the proteins.

In embodiments, modifying the function and/or expression of an enzyme described herein (e.g., ALKBH5, METTL3 and METTL14) comprises decreasing expression of the enzyme, or inhibiting one or more functions of the enzyme.

In embodiments, expression of an enzyme described herein is inhibited. Inhibition of expression of an enzyme (or any other protein) can be achieved using methods known in the art. In embodiments, expression is inhibited by inhibiting translation of mRNA encoding the protein. In embodiments, the mRNA encoding the protein is degraded. In this regard, in non-limiting embodiments, RNA interference (RNAi)-mediated silencing and/or reducing mRNA encoding an enzyme described herein is performed. In embodiments, this is achieved by delivery of any suitable RNAi agent. In embodiments, an siRNA-based approach is used. This can be performed by introducing and/or expressing one or more suitable short hairpin RNAs (shRNA) in the cells. shRNA is an RNA molecule that contains a sense strand, antisense strand, and a short loop sequence between the sense and antisense fragments. shRNA is exported into the cytoplasm where it is processed by dicer into short interfering RNA (siRNA). siRNA are 21-23 nucleotide double-stranded RNA molecules that are recognized by the RNA-induced silencing complex (RISC). Once incorporated into RISC, siRNA facilitate cleavage and degradation of targeted mRNA. Thus, for use in RNAi mediated silencing or downregulation of ALKBH5 expression as described herein, siRNA, shRNA, or miRNA can be used. In alternative embodiments, a functional RNA, such as a ribozyme is used. In embodiments, the ribozyme comprises a hammerhead ribozyme, a hairpin ribozyme, or a Hepatitis Delta Virus ribozyme. In related embodiments, a microRNA (miRNA) adapted to target the relevant mRNA can be used.

In alternative embodiments, one or more small molecule inhibitors of ALKBH5, METTL3 and METTL14 can be used in methods of this disclosure. In connection this, the present disclosure includes a proof of principle demonstrating that ALKBH5 function can be inhibited by a small molecule. To show this, a small molecule inhibitor of 2-oxoglutarate-dependent oxygenases (a class of enzymes that includes ALKBH5), is shown to reduce IFNB1 mRNA accumulation in response to exposing primary human fibroblasts to ds DNA, which, as described herein, is a known inducer of type I interferon and inflammatory responses. This demonstration was performed using a compound referred to in the art as Rhein, which has the following structure:

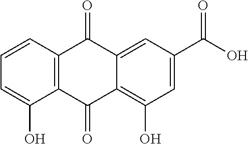 [SMILES: O=C(O)c1cc(O)c2c(c1)C(=O)c1cccc(O)c1C2=O]

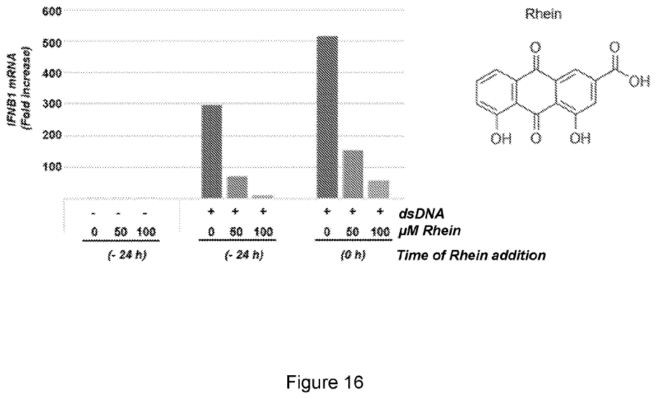

Results from testing this compound are provided in FIG. 16. As shown in FIG. 16, Rhein was tested in primary human fibroblasts, which were transfected with or without dsDNA. After 9 hours, IFNB1 mRNA accumulation was measured by RT-PCR. Cultures were treated with vehicle (DMSO, 0 μM Rhein) or the indicated concentrations of Rhein per FIG. 16, either 24 hours prior to transfection (−24 h) or at the time of transfection (0 h). As can be seen from FIG. 16, exposing dsDNA positive cells to Rhein causes a dose-dependent inhibition of IFNB1 mRNA production. Accordingly, when these data are taken in the context of the RNAi-mediated inhibition of ALKBH5 expression results described below, which demonstrate a similar reduction in IFNB1 mRNA, the disclosure supports the use of any agent that can inhibit the expression and/or function of ALKBH5. Further, the disclosure provides for use of rationally designed ALKBH5 inhibitors, or those identified by a screening approach that is further described herein. For rational drug design, the crystal structure ALKBH5 is known and can be used as a basis for designing small molecules that target enzymatically important residues. (See, for example, Feng, et al. Crystal Structures of the Human RNA Demethylase Alkbh5 Reveal Basis for Substrate Recognition, (2014) The Journal of Biological Chemistry, 289, 11571-11583, the disclosure from which is incorporated herein by reference). Thus, based at least in part on this crystal structure, compounds that function as inhibitors of ALKBH5 can be designed and tested for ALKBH5 inhibition. For example, in non-limiting embodiments, derivatives of Rhein, or other actual or predicted small molecules, can be produced and tested for enzymatic inhibition function, and can be modified to exhibit improved pharmacokinetic properties, such as absorption, bioavailability, distribution, metabolism, excretion, selectivity, and specificity.

Determining inhibition of ALKBH5, and thus identifying compounds for use in ALKBH5 inhibition, can be performed, given the benefit of this disclosure, by determining whether or not a compound inhibits activation of the type I IFN pathway. Additionally or alternatively, determining inhibition of ALKBH5 can be assessed by determining a reduction in dsDNA-induced IFNB1 mRNA accumulation. In embodiments, IFNB1 mRNA accumulation can be induced in test cells, such as by treating cells to provoke formation of cytoplasmic dsDNA, or introducing cytoplasmic dsDNA, including but not limited to infection by a DNA virus, or using cells that have known cytoplasmic dsDNA to establish a value for IFNB1 mRNA. Inhibition can therefore be identified by determining a value for IFNB1 mRNA that is lower than an established control value. Such tests could be determined, for example, in a high-throughput screening approach using in vitro cell cultures. Changes in secretion of IFNB1 can also be measured in such tests. The same approach can be adapted for identifying inhibitors of METTL3 and METTL14, but in this approach an increase in IFNB1 is indicative of enzymatic inhibition. Further, methylation patterns of m⁶A mRNA can be analyzed to identify agents that modulate the activity of ALKBH5, or METTL3/14. Further still, inhibiting expression of ALKBH5 or METTL3/14 impacts expression of numerous genes, as further described herein, and in the accompanying figures. Expression of one or any of these genes can also be measured to assess whether or not compound effect the function of these enzymes. Thus, in embodiments, it is expected that some or all cellular genes regulated together with IFNB1 mRNA described in the Figures would likewise be impacted by interfering with METTL3/14 or ALKBH5 expression. In further embodiments, test compounds could be assessed to determine their effects on senescence-associated characteristics that are associated with the function of any enzyme described herein.

In embodiments, the disclosure relates to inhibitors of ALKBH5, or inhibition of its expression, to treat autoimmune diseases, or inflammatory diseases, or a combination thereof. Thus, the disclosure facilitates reducing expression if IFNB1 in individuals in need of treatment for such conditions. In embodiments, the individual has been diagnosed with or is suspected of having an autoimmune disease, which can include but is not necessarily limited to systemic lupus erythematosus, rheumatoid arthritis, chronic inflammation, and any of a number of interferonopathies, such as such as Aicardi-Goutieres syndrome. In connection with these utilities for the present disclosure, it is known in the art that self-DNA can be causative of autoimmune diseases. (See, for example, Gao et al., Activation of cyclic GMP-AMP synthase by self-DNA causes autoimmune disease, Proc Natl Acad Sci USA. 2015 Oct. 20; 112(42):E5699-705, and Vincent et al., Small molecule inhibition of cGAS reduces interferon expression in primary macrophages from autoimmune mice, Nature Communications, 8: 750, DOI: 10.10381s41467-017-00833-9, the disclosures of which are incorporated herein by reference). Further, Type I IFN is implicated in the pathogenesis of systemic lupus erythematosus and interferonopathies. (See, for example, An, et al. Expression of Cyclic GMP-AMP Synthase in Patients With Systemic Lupus Erythematosus, ARTHRITIS & RHEUMATOLOGY, Vol. 69, No. 4, April 2017, pp 800-807, the disclosure of which is incorporated herein by reference). Additional and non-limiting examples of specific disorders include celiac disease, Crohn's disease, diabetes mellitus type 1, autoimmune encephalitis, eosinophilic fasciitis, eosinophilic gastroenteritis, eosinophilic esophagitis, multiple sclerosis (MS), including but not limited to Relapsing-Remitting MS (RRMS), Secondary-Progressive MS (SPIVS), Primary-Progressive MS (PPMS), and Progressive-Relapsing MS (PRMS), or gastritis, Graves' disease, hypogammaglobulinemia, idiopathic inflammatory demyelinating diseases, thrombocytopenic purpura, myasthenia gravis, pernicious anemia, psoriasis, Sjögren's syndrome, and ulcerative colitis, graft versus host disease (GVHD). In further embodiments, an individual treated with an ALKBH5 inhibitor or suppressor may have any disorder that is characterized by type II, III or IV hypersensitivity.

In embodiments, treatment of an individual with an agent that reduces expression and/or inhibits ALKBH5 function can be combined with an immune modulator. For example, an immunosuppressant agent may be used. In embodiments, the immunosuppressive agent may comprises an mTOR inhibitor, such as rapamycin, or a rapolog. In embodiments, the immunosuppressant comprises a calcineurin inhibitor, such as tacrolimus or cyclosporine, or a corticosteroid, such as cortisone prednisolone, prednisone, and triamcinolone.

In other embodiments, an individual is treated with an agent that reduces expression and/or inhibits the function of METTL3, or METTL14, or both, to thereby produce an increase in IFNB1 mRNA and/or IFNβ cytokine. In embodiments, the individual has a DNA virus or microbial infection. In embodiments, an approach of this disclosure is therefore suitable for treating an individual who is infected with a Poxvirus, a Herpesvirus, a Hepatitis virus, an Adenovirus, a Polyomavirus, or a Papillomavirus. In embodiments, an approach of this disclosure for treating a viral infection can be combined with treatment of the individual with any suitable anti-viral drug, examples of which include but are not necessarily limited to Acyclovir, Ganciclovir, Valganciclovir, Valacyclovir, Idoxuridine, Trifulodine, Vidar, Famciclovir, Penciclovir, Boceprevir, Ledipasvir, Ombitasvir, Paritaprevir, Simeprevir and Telaprevir.

In embodiments, an individual is treated with an agent that reduces expression and/or inhibits the function of METTL3 and METTL14 to thereby produce an increase in IFNB1 mRNA and/or IFNβ cytokine, when the individual has a microbial infection, such by as a non-viral intracellular pathogenic microbe. In embodiments, the intracellular microbial pathogen is a bacteria, or a fungus. In embodiments, the bacterial infection is caused by an obligate intracellular parasite (e.g., any member of Chlamydophila, Ehrlichia, Rickettsia). Additionally, certain pathogenic types of Salmonella, Neisseria, Brucella, Mycobacterium, Nocardia, Listeria, Francisella, Legionella, and Yersinia pesti may be present in intracellular infections, and treating such infections is also encompassed by this disclosure. In embodiments, an individual who is immunocompromised for any reason is treated with an agent that reduces expression and/or inhibits the function of METTL3 and METTL14. In embodiments, the immunocompromised individual does not produce IFN, or produced less than a suitable amount of IFN.

In embodiments, the individual has a fungal infection. Without intending to be constrained by any particular theory, it is considered that fungal DNA may be present in cytoplasm of human cells, under certain conditions, but this is not believed to essential for implementing a method of the disclosure. In embodiments, the fungal infection is by any member of Candida, Aspergillus, Cryptococcus, Histoplasma, Pneumocystis, or Stachybotrys.

Treatment of bacteria or fungal infections according to this disclosure can be combined with any suitable antibacterial or antifungal agents. In embodiments, suitable antibiotics include but are not limited to members of classes such as aminoglycosides, beta lactams (with or without beta lactamase inhibitor such as clavulanic acid), macrolides, glycopeptides, polypeptides, cephalosporins, lincosamides, ketolides, rifampicin, polyketides, carbapenem, pleuromutilin, quinolones, streptogranins, oxazolidinones, lipopeptides, etc. In embodiments, anti-fungal agents include but are not limited to flucytosine, imidazoles, polyenes, triazoles, griseofulvin, ciclopirox, terbinafine, and caspofungin.

In embodiments, an individual is treated with an agent that reduces expression and/or inhibits the function of METTL3 and METTL14 to thereby produce an increase in IFNB1 mRNA and IFNβ cytokine, wherein the individual has cancer. In embodiments, the individual has been diagnosed with any type of cancer. In embodiments, the cancer is a solid or liquid tumor. In embodiments, the cancer is renal cell carcinoma, breast cancer, prostate cancer, pancreatic cancer, lung cancer, liver cancer, ovarian cancer, cervical cancer, colon cancer, esophageal cancer, stomach cancer, bladder cancer, brain cancer, testicular cancer, head and neck cancer, melanoma or another skin cancer, any sarcoma, including but not limited to fibrosarcoma, angiosarcoma, adenocarcinoma, and rhabdomyosarcoma, and any blood cancer, including all types of leukemia, lymphoma, and myeloma.

In embodiments, treatment with an enzyme inhibitor described herein is combined with a chemotherapeutic agent, including but not necessarily limited to an immune checkpoint inhibitor. In embodiments, the checkpoint inhibitors comprise an anti-programmed cell death protein 1 (anti-PD-1) checkpoint inhibitor, or an anti-Cytotoxic T-lymphocyte-associated protein 4 (anti-CTLA-4) checkpoint inhibitor. There are numerous such checkpoint inhibitors known in the art. For example, anti-PD-1 agents include Pembrolizumab and Nivolumab. An anti-PD-L1 example is Avelumab. An anti-CTLA-4 example is Ipilimumab.

In embodiments, an agent of this disclosure, such as agents used to inhibit the expression and/or function of ALKBH5, METTL3, or METTL14, is administered in a therapeutically effective amount. For any such agent, the therapeutically effective amount, e.g., a dose, can be estimated initially either in cell culture assays or in animal models. An animal model can also be used to determine a suitable concentration range, and route of administration. Such information can then be used to determine useful doses and routes for administration in humans. A precise dosage can be selected by the individual physician in view of the patient to be treated. Dosage and administration can be adjusted to provide sufficient levels of the active moiety or to maintain the desired effect. Additional factors which may be taken into account include the severity and type of the disease state, age, weight and gender of the patient, desired duration of treatment, method of administration, time and frequency of administration, drug combination(s), reaction sensitivities, and tolerance/response to therapy. A therapeutically effective amount is an amount that reduces one or more signs or symptoms of a disease, and/or reduces the severity of the disease. A therapeutically effective amount may also inhibit or prevent the onset of a disease, or a disease relapse.

In embodiments, an enzyme inhibitor used in methods of this disclosure is administered to a patient in a sufficient quantity to achieve a peak plasma concentration in a therapeutic target range. In embodiments, an enzyme inhibitor is used to achieve a plasma concentration of between 0.1 nM and 1 μM, inclusive, and including all numbers there between to the first decimal point. These or other plasma concentrations can for an individual agent or patient can be determined by pharmacodynamics studies that will be apparent to those skilled in the art, given the benefit of the present disclosure.

Administration of compositions comprising agents that modulate the expression and/or function of the enzymes as described herein can be carried out using any suitable route of administration known in the art. For example, the compositions may be administered via intravenous, intramuscular, intraperitoneal, intracerobrospinal, subcutaneous, intra-articular, intrasynovial, oral, topical, or inhalation routes, depending on the particular condition being treated. The compositions may be administered parenterally or enterically. The compositions may be introduced as a single administration or as multiple administrations or may be introduced in a continuous manner over a period of time. For example, the administration(s) can be a pre-specified number of administrations or daily, weekly or monthly administrations, which may be continuous or intermittent, as may be therapeutically indicated.

Enzyme inhibitors or other suitable agents for use in embodiments of the present disclosure can be provided in pharmaceutical compositions by combining them with any suitable pharmaceutically acceptable carriers, excipients and/or stabilizers. Examples of pharmaceutically acceptable carriers, excipients and stabilizer can be found in Remington: The Science and Practice of Pharmacy (2005) 21st Edition, Philadelphia, Pa. Lippincott Williams & Wilkins, the disclosure of which is incorporated herein by reference. For example, suitable carriers include excipients, or stabilizers which are nontoxic to recipients at the dosages and concentrations employed, and include buffers such as acetate, Tris, phosphate, citrate, and other organic acids; antioxidants including ascorbic acid and methionine; preservatives such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; and/or non-ionic surfactants such as Tween or polyethylene glycol (PEG).

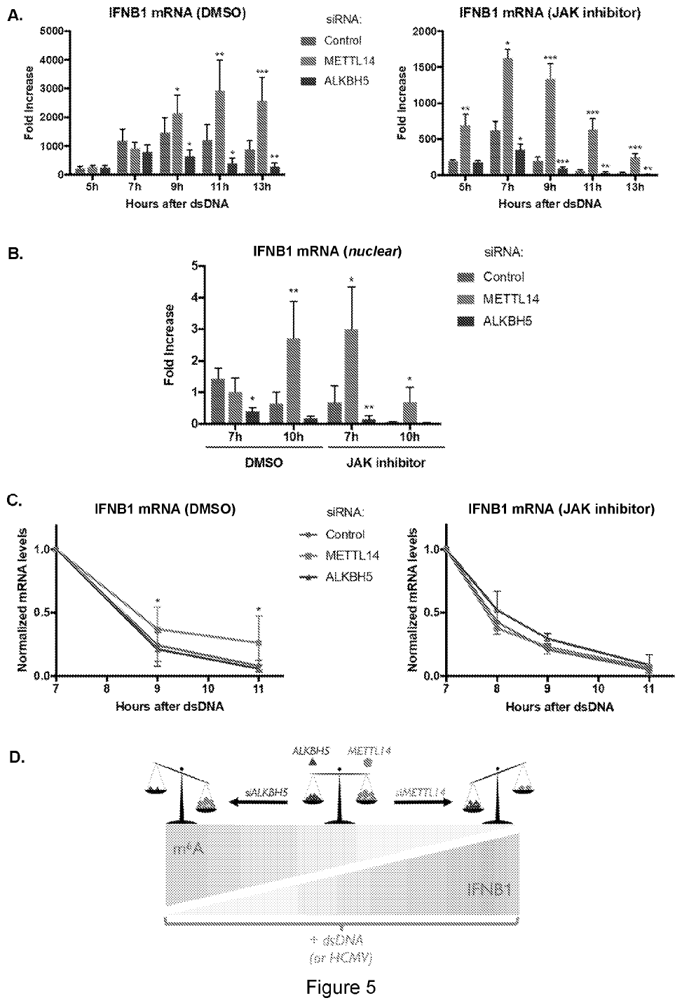

The Examples below, which are not intended to limit the invention, demonstrate that host enzymes that install and remove m⁶A increase in abundance in response to HCMV infection and regulate virus replication. While depleting the ALKBH5 demethylase stimulated viral protein accumulation and HCMV reproduction, interfering with the METTL3/14 methylase had the opposite effect. The impact of the cellular m⁶A modification machinery on HCMV growth resulted from differential IFNβ cytokine production, as IFNB1 mRNA accumulation was stimulated by METTL14-depletion and restricted by ALKBH5-depletion (FIG. 5D). Moreover, IFNβ cytokine production in uninfected cells treated with dsDNA was likewise regulated by METTL14 and ALKBH5 and IFNB1 mRNA was enriched in an m⁶A RNA-containing fraction. Additionally, genome-wide profiling of cells exposed to dsDNA following ALKBH5-depletion identified differentially-expressed genes regulating anti-viral immune responses, while METTL14-depletion altered pathways involving metabolic reprogramming, stress responses, and aging. Thus, by analyzing how the METTL14 m⁶A methyltransferase subunit and the ALKBH5 demethylase control HCMV replication, the disclosure reveals a fundamental mechanism whereby host cell intrinsic immune responses to dsDNA in uninfected cells are epitranscriptomically controlled by m⁶A RNA modification.

While cellular m⁶A modification enzymes control productive replication of the beta-herpesvirus HCMV by influencing IFNB1 accumulation and IFNβ cytokine production, effects on HSV-1, an alpha-herpesvirus subfamily member, and the prototypical poxvirus Vaccinia were not detected. Without intending to be bound by any particular theory, this might reflect differences in cell intrinsic anti-viral responses. Notably, cellular protein synthesis proceeds in HCMV-infected cells, in marked contrast to the strong suppression of ongoing host proteins synthesis observed in cells infected with HSV-1 or Vaccinia that effectively limit ISG expression (Zhu et al, 1997; Ishikawa et al., 2009). Replication of other viruses, including HIV and influenza A virus, is stimulated by the METTL3/14 m⁶A writer complex and may also in part reflect reduced IFNB1 induction (Courtney et al., 2017).

In embodiments, the disclosure provides compositions and methods for enhancing virus replication, and thus may be used to increase, for example, viral particle production for any purposes, including but not limited to producing viral particles for use in vaccines. In embodiments, the disclosure thus includes inhibiting the expression and/or function of ALKBH5 in cells, such as a cell culture, that is infected and/or has been transduced with a virus, allowing increased production of the virus, relative to production of the virus in cells in which ALKBH5 expression and/or function is not reduced, and separating the virus and/or viral particles from the cells. In embodiments, the viral particles are infectious, non-pathogenic viral particles for use as a vaccine. In embodiments, the disclosure comprises using cells in such a process, wherein ALKBH5 expression is knocked out in the cells, such as by using a CRISPR-gene editing approach. Such cells and use of such cells for producing viruses are included in the disclosure. In embodiments, the cells are mammalian cells, such as human cells. In embodiments, the cells are non-human primate cells.

Data presented in this disclosure indicates that IFNB1 mRNA is enriched in an m⁶A-containing mRNA fraction and its accumulation is controlled by the m⁶A methyltransferase subunit METTL14 and the demethylase ALKBH5. Depleting METTL14 increased IFNB1 mRNA stability, which is known to be controlled by ELAVL1/HuR-binding to AU-rich sequences in the 3′-UTR. Nucleic acid sensing pathways are thought to control ELAVL1/HuR activity (Herdy et al., 2015; Takiuichi, 2015), potentially explaining why IFNB1 mRNA stability was enhanced by METTL14-depletion in a manner dependent upon IFN signaling (FIG. 5C). The impact of METTL14 on IFNB1 mRNA decay is consistent with the reported role for m⁶A in genome-wide mRNA stability (Wang et al, 2014; Ke et al, 2017). One difference being that unlike the majority of cellular mRNAs, which are spliced, IFNB1 mRNA does not contain an intron.

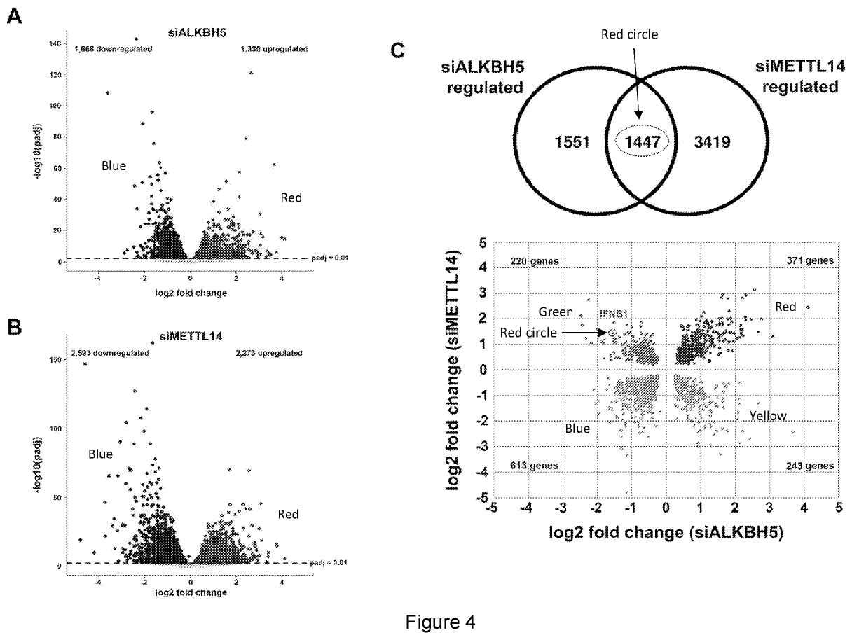

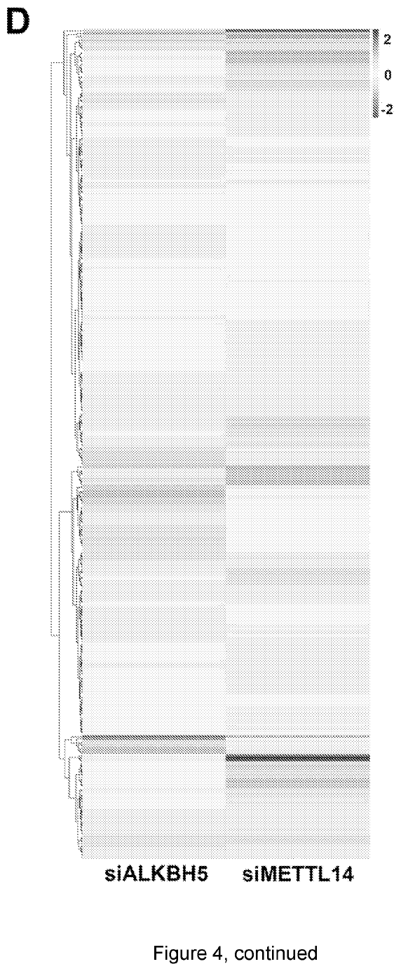

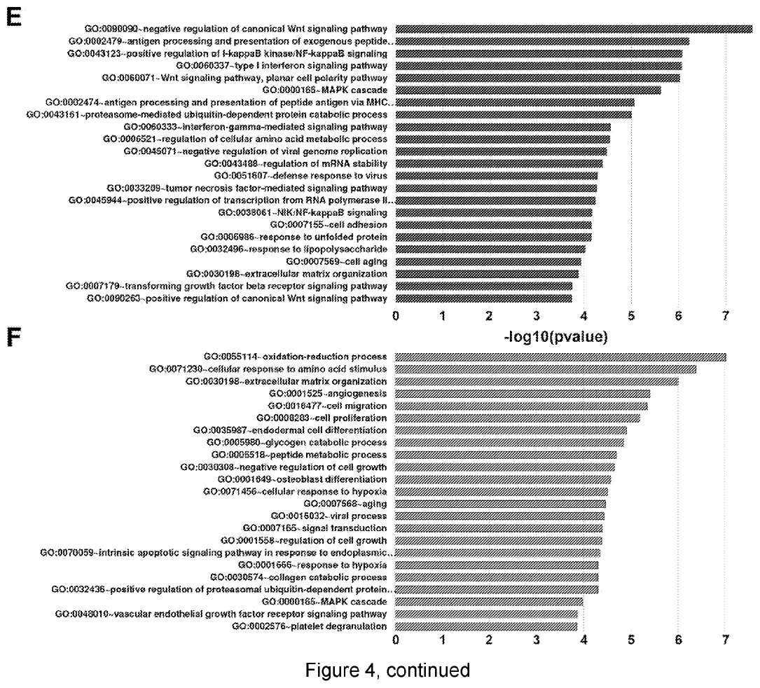

Unexpectedly, METTL14-depletion also stimulated EU incorporation into nascent, nuclear IFNB1 RNA following dsDNA treatment, whereas ALKBH5-depletion repressed it (FIG. 5B). In addition, the overall magnitude of differences in EU-incorporation into nascent RNA greatly exceeded any differences detected in IFNB1 mRNA stability (compare FIG. 5B to 5C). Without intending to be constrained by any particular theory, this suggests that ALKBH5 and METTL14 have a greater impact on IFNB1 mRNA production than decay. As differential IRF3 or NF-κB activation or the subcellular distribution of the cellular m⁶A machinery could not account for the observed changes in IFNB1 mRNA abundance, limiting m⁶A installation might stimulate mRNA biogenesis at the level of transcription. Again, without intending to be bound by theory, this could occur upstream of IFNB1 mRNA transcription. Pathway analysis of ALKBH5 and METTL14-depleted NHDFs treated with dsDNA implicates biological processes including mRNA biogenesis and decay as potential effectors shaping genome-wide responses that impact subsequent IFNB1 mRNA biogenesis. (FIGS. 4, 9, 11-12). Alternatively, co-transcriptional deposition of m⁶A onto nascent IFNB1 mRNA might directly influence transcription initiation or elongation.

Genome-wide profiling data from uninfected NHDFs revealed responses to dsDNA exposure are regulated by METTL14 and ALKBH5. In particular, opposing, reciprocal effects on expression of 463 genes including IFNB1, approximately 32% of genes regulated by both METTL14 and ALKBH5-depletion, was observed in response to dsDNA. In contrast, the effects of ALKBH5 or METTL14-depletion were not entirely reciprocal genome-wide. 68% of genes responsive to both ALKBH5 and METTL14 depletion were co-regulated in the same direction. In addition to targeting an identical cohort of co-regulated and reciprocally regulated genes upon dsDNA treatment, METTL14 and ALKBH5 also target discrete gene sets with little overlap. This indicates that while ALKBH5 and METTL14 regulate innate immune responses via IFNB1 mRNA induction, they largely control different, non-overlapping biological processes.

In addition to infection biology, roles for cytoplasmic dsDNA signaling from non-microbial sources have emerged. The cytoplasmic DNA sensor cGAS controls senescence and senescent cells express greater levels of IFNB1 and proinflammatory cytokines as part of a senescence-associated secretory phenotype (SASP) (Takahashi et al., 2018; Li & Chen, 2018). By controlling IFNB1, cellular m⁶A modification enzymes might also impact aging, senescence and the pathogenic consequences of overcoming senescence associated with cancer. As cytoplasmic dsDNA signaling is also a trigger for inflammatory diseases, epitranscriptomic regulation of responses triggered by dsDNA could broadly impact pathophysiology associated with systemic lupus erythematosus, rheumatoid arthritis, chronic inflammation and interferonopathies (Gao et al., 2015; Chen et al., 2016; Crowl et al., 2017; Yan, 2017; Li et al., 2018). Thus, modulating IFNB1 as described herein is expected to be broadly applicable to prophylaxis and/or therapy for a wide array of disorders that involve pathological deregulation of inflammatory cytokines, such as IFNB1.

The following Examples are intended to illustrate but not limit the disclosure.

### Example 1

Regulation of HCMV Replication by Host m⁶A Modification Enzymes

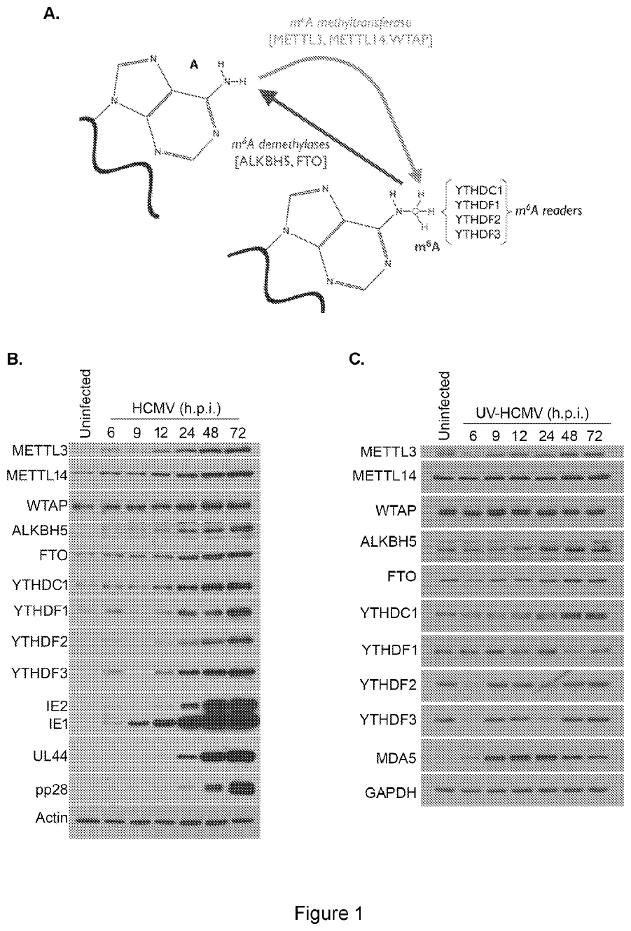

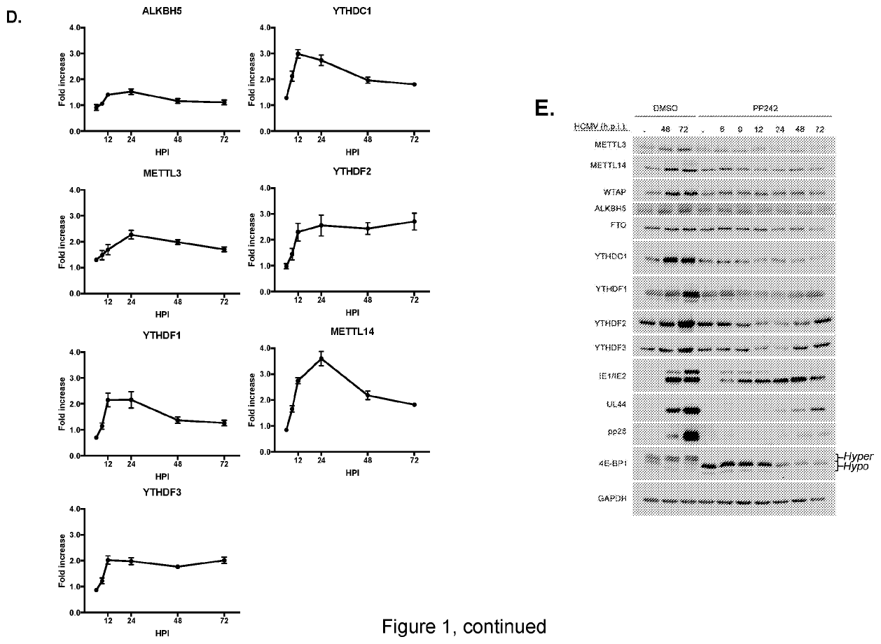

Since host protein synthesis is not impaired by human cytomegalovirus (HCMV) infection (McKinney et al., 2014), we could examine how cellular functions required for m⁶A RNA modification responded to HCMV and influenced virus reproduction. Compared to uninfected primary human fibroblasts (NHDFs), HCMV infection resulted in accumulation of m⁶A methyltransferase subunits METTL3/14, m⁶A readers YTHDC1, YTHDF1,2,3 and the demethylases ALKBH5 and FTO between 12-72 h post-infection (FIG. 1B). This most closely coincided temporally with accumulation of the representative HCMV early (E) protein UL44 (FIG. 1B) and was severely curtailed following infection with UV-inactivated HCMV indicating that virus-induced accumulation of cellular m⁶A effector polypeptides was largely dependent upon viral gene expression (FIG. 1C). These modest increases in abundance observed 48-72 h after infection with UV inactivated HCMV are unlikely to result from incomplete UV inactivation as they were not observed for all m⁶A effector proteins. It could, however, indicate a secondary level of induction independent of virus gene expression.

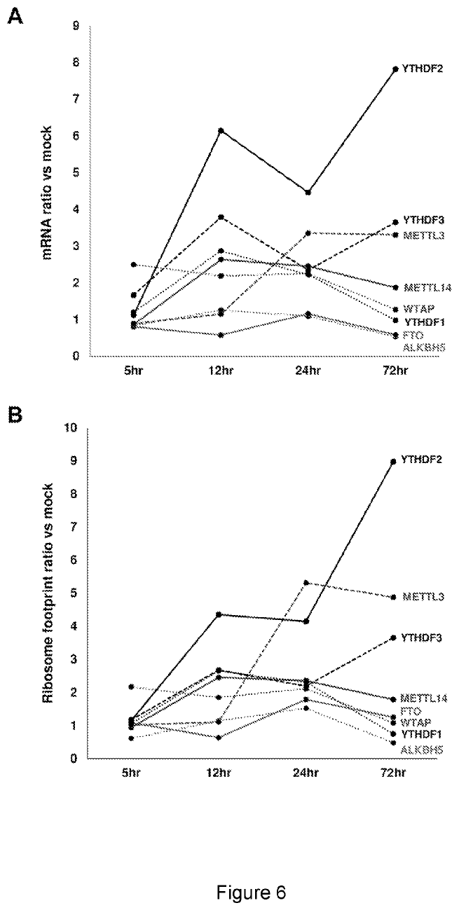

In response to viral gene expression, a less than two-fold increase in ALKBH5 mRNA was observed over time (FIG. 1D). Whereas METTL3, YTHDF1 and YTHDF3 mRNA increased approximately two-fold, a three to four-fold increase in mRNA levels was detected for YTHDC1, YTHDF2 and METTTL14 (FIG. 1D). Finally, the HCMV-induced increase in cellular m⁶A effectors was repressed by treatment with the mechanistic target of rapamycin (mTOR) active-site inhibitor PP242 (FIG. 1E). This indicates that the increase in m⁶A writers, readers and erasers was dependent upon mTOR activation in HCMV-infected cells. The HCMV UL38 protein, which is expressed at early times post-infection, activates mTOR-complex 1 (mTORC1) and has been shown to regulate cap-dependent translation of host mRNAs in HCMV-infected cells (McKinney et al, 2012; 2014). Taken together, these data suggest that the increase in cellular m⁶A-effector proteins induced by HCMV is in part regulated by changes in mRNA abundance and cap-dependent mRNA translation. Similar changes in mRNA abundance and mRNA translation inferred from ribosome profiling have been reported by others (Tirosh et al., 2015) in cells infected with a different HCMV strain (FIG. 6).

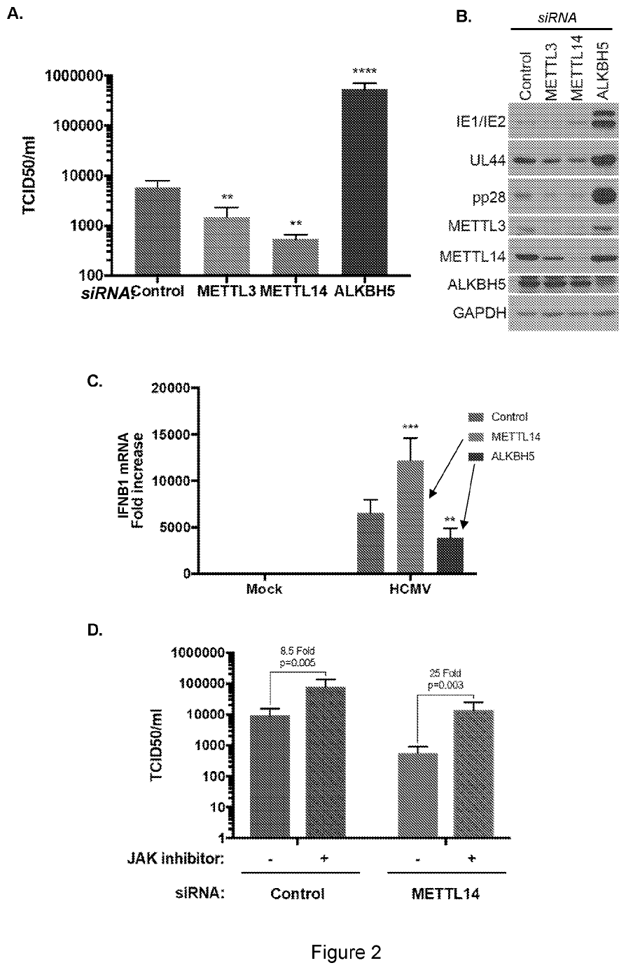

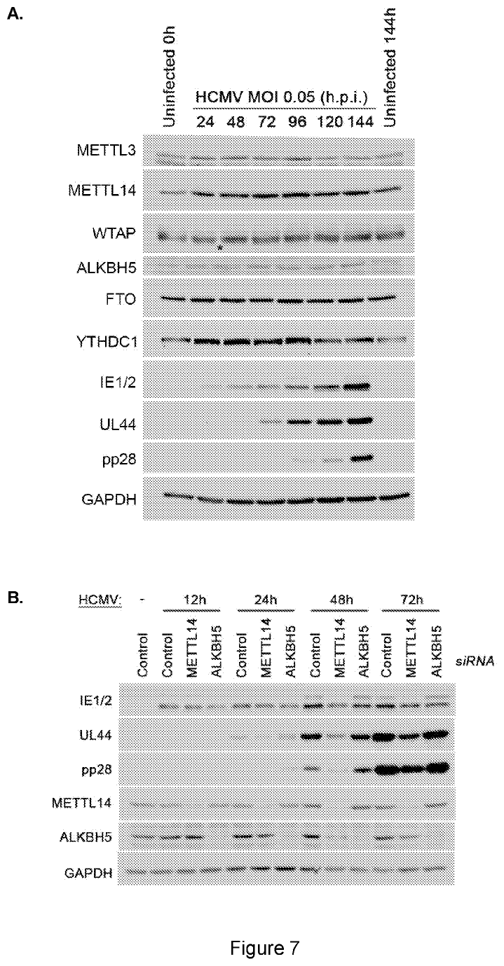

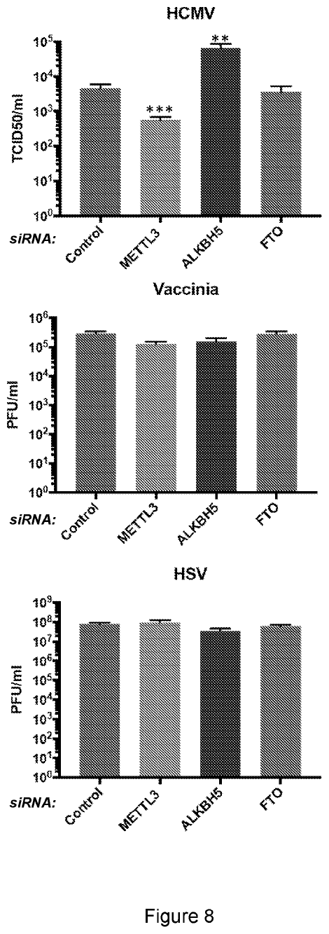

To determine how host m⁶A writers and erasers influenced virus replication, cultures were treated with siRNA specific for METTL3, METTL14, ALKBH5 or control, non-silencing siRNA. Subsequently, siRNA-treated NHDFs were mock-infected or infected with HCMV at a low multiplicity of infection, which allows for measurement of virus reproduction and spread through the culture. Compared to non-silencing siRNA-treated cultures, METTL3/14-specific siRNAs inhibited HCMV reproduction up to 11-fold (FIG. 2A), and reduced accumulation of representative virus immediate-early (IE) E and late (L) proteins (FIG. 2B). Even under these conditions where approximately one in 20 cells were initially infected, a virus-induced increase in METTL14 and ALKBH5 abundance remained detectable (FIG. 7A), albeit modest in comparison to the increase observed at greater viral inoculums where the majority of cells were infected (FIG. 1B). In addition, a greater reduction in HCMV reproduction was observed with METTL14 siRNA compared to METTL3 siRNA-treated cultures. As both METTL3 and METTL14 are components contained within a multi-subunit methyltransferase (Liu et al, 2014), this likely reflects the larger reduction in METTL3 and METTL14 levels achieved using METTL14 siRNA compared to METTL3 siRNA (FIG. 2B). Importantly, METTL3 and METTL14 siRNAs did not detectably change ALKBH5 or GAPDH levels (FIG. 2B). METTL14-depletion similarly reduced accumulation of representative HCMV immediate-early, early and late proteins over time when high multiplicity infections were performed (FIG. 7B). By contrast, ALKBH5-siRNA enhanced virus protein accumulation and stimulated virus reproduction and spread by nearly 100-fold following infection at low multiplicity (FIG. 2A,B). ALKBH5-depletion did not detectably increase representative levels of HCMV IE1/2 (immediate-early), UL44 (early) or pp28 (late) proteins in cells infected at high multiplicity (FIG. 7B). Depleting the demethylase FTO did not detectably stimulate HCMV reproduction (FIG. 8). The selective effect of ALKBH5 on HCMV reproduction (FIG. 8) is consistent with the restricted capacity of FTO to demethylate internal m⁶A residues and its preference for m⁶Aₘ substrates (Mauer et al, 2017). Finally, depleting METTL3 or ALKBH5 did not measurably impact replication of other DNA viruses including HSV-1 or vaccinia virus (FIG. 8).

### Example 2

Cellular m⁶A Modification Enzymes Regulate IFNB1 mRNA Accumulation in HCMV-Infected Cells

To determine if host functions regulating m⁶A modification controlled anti-viral immune responses, IFNB1 mRNA abundance was measured in HCMV-infected cells treated with non-silencing siRNA or siRNA specific for METTL14 or ALKBH5. While IFNB1 mRNA levels were significantly elevated in cultures treated with METTL14 siRNA relative to non-silencing siRNA, ALKBH5 siRNA-treated cultures contained reduced IFNB1 mRNA (FIG. 2C). This suggested that greater HCMV reproduction in ALKBH5-depleted cells might result from less IFNβ cytokine production, while reduced virus replication in METTL14-depleted cultures resulted from greater IFNβ production. To test this hypothesis, the involvement of IFN signaling in the inhibition of HCMV replication by METTL14-depletion was examined. NHDFs treated with control, non-silencing siRNA or METTL14 siRNA were infected with HCMV and virus replication was measured in the presence or absence of a Janus kinase (JAK) inhibitor, which inhibits JAK signaling downstream of the type I IFN receptor. While HCMV replication remained inhibited by METTL14-siRNA compared to control siRNA, JAK inhibitor treatment to a large extent restored HCMV reproduction in METTL14 siRNA-treated cultures to levels observed in cultures treated with control siRNA (FIG. 2D). By contrast, addition of JAK inhibitor to control siRNA-treated cultures resulted in a small, less than 10-fold increase (FIG. 2D). This indicates that restriction of HCMV reproduction by METTL14 siRNA likely resulted from IFNβ production and subsequent signaling dependent upon JAK-STAT.

### Example 3

m⁶A Modification Enzymes and Reader Proteins Control Cell Intrinsic Innate Responses to dsDNA in Uninfected Cells

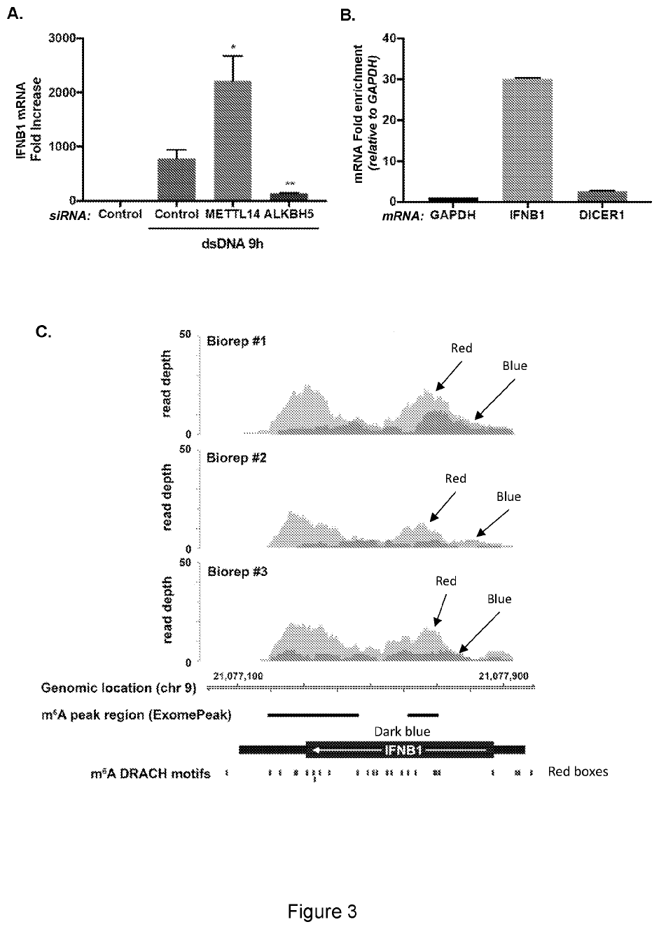

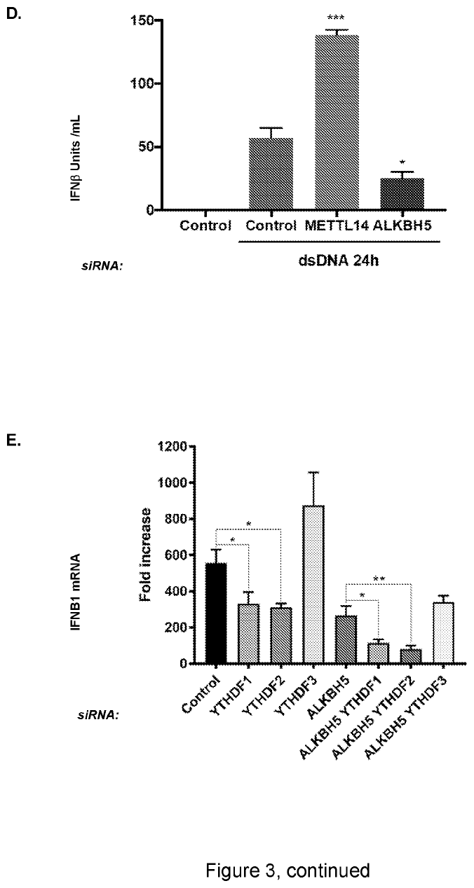

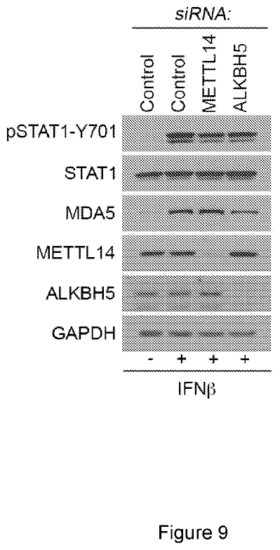

Since HCMV is a dsDNA virus, and dsDNA alone is sufficient to trigger IFNβ production (Chen et al., 2016), it was conceivable that the control of IFNβ cytokine production by m⁶A modifying enzymes might not be restricted to virus-infected cells. To test this possibility the capacity of cellular m⁶A writers and erasers to regulate innate IFNβ production in uninfected cells was evaluated. Addition of dsDNA effectively stimulated IFNB1 mRNA accumulation in non-silencing siRNA-treated cultures (FIG. 3A). Unexpectedly, METTL14-depletion significantly enhanced IFNB1 mRNA accumulation while ALKBH5-depletion effectively limited IFNB1 mRNA accumulation in uninfected cells (FIG. 3A). This suggested that IFNB1 mRNA might be m⁶A-modified. Indeed, compared to GAPDH and DICER, a cellular mRNA known to be m⁶A-modified, IFNB1 mRNA was substantially enriched in an RNA fraction immunoprecipitated from dsDNA-treated NHDFs using an m⁶A-specific antibody (FIG. 3B). Analysis of m⁶A-enriched RNA by RNA-seq identified m⁶A peaks mapping over two discrete IFNB1 mRNA segments (FIG. 3C) that each contain consensus m⁶A acceptor sites (Linder et al., 2015). While one cluster (supported by 2/3 biological replicates) maps entirely within the IFNB1 coding sequences, the second (supported by 3/3 biological replicates) includes the junction between the open reading frame and 3′-UTR (FIG. 3C). Others have similarly found methylated mRNA residues enriched within UTRs near stop codons (Dominissini et al., 2012; Meyer et al, 2012). Finally, depleting METTL14 or ALKBH5 did not detectably alter the responsiveness of target cells to exogenously added IFNβ as measured by STAT Y701 phosphorylation or prevent accumulation of a representative ISG (FIG. 9). Instead, the observed changes in IFNB1 mRNA levels reflected differential production of functional IFNβ cytokine (FIG. 3D).

In addition to m⁶A writers and erasers, reader proteins that recognize m⁶A modified RNA could potentially impact IFNβ production in response to dsDNA in uninfected cells. Compared to NHDFs treated with control, non-silencing siRNA, depleting m⁶A readers YTHDF1 or YTHDF2 reduced IFNB1 mRNA induction in response to dsDNA (FIG. 3E). By contrast, IFNB1 mRNA levels were not significantly different in response to dsDNA following m⁶A reader YTHDF3-depletion (FIG. 3E). This indicated that depleting YTHDF1 or YTHDF2 was similar to depleting the ALKBH5 demethylase as both suppressed IFNB1 mRNA induction by dsDNA (FIG. 3E). Remarkably, co-depletion of ALKBH5 together with YTHDF1 or YTHDF2 exacerbated the reduction in dsDNA-induced IFNB1 mRNA accumulation to levels below that observed when cells were singly depleted for either ALKBH5, YTHDF1, or YTHDF2 (FIG. 3E). This demonstrates a synthetic genetic interaction between ALKBH5 demethylase and the m⁶A readers YTHDF1 and YTHDF2. It further indicates that these two m⁶A readers limit the extent to which ALKBH5-depletion suppresses IFNβ production and is consistent with the m⁶A demethylase and readers acting in the same pathway to regulate IFNB1 mRNA levels.

### Example 4

Regulation of Genome-Wide Responses to dsDNA by METTL14 and ALKBH5

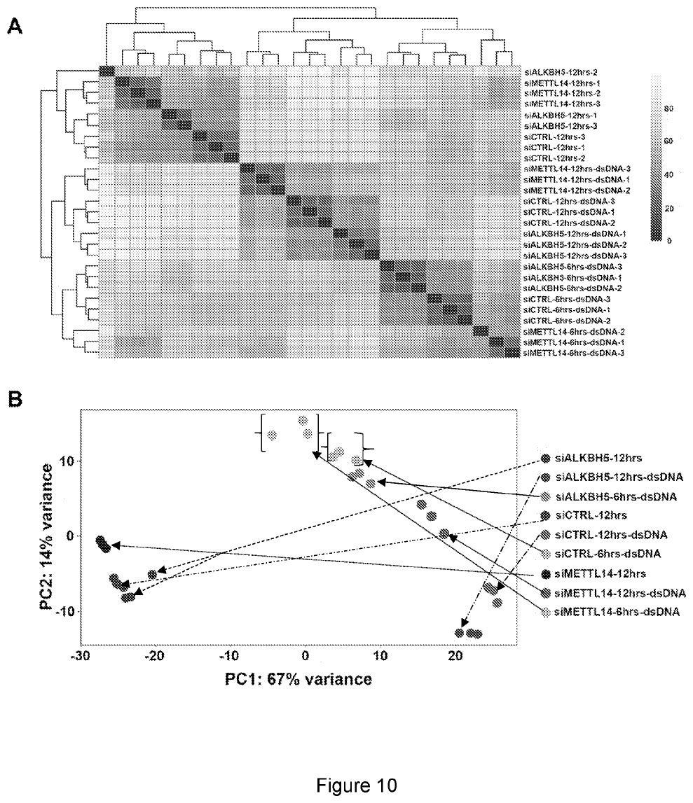

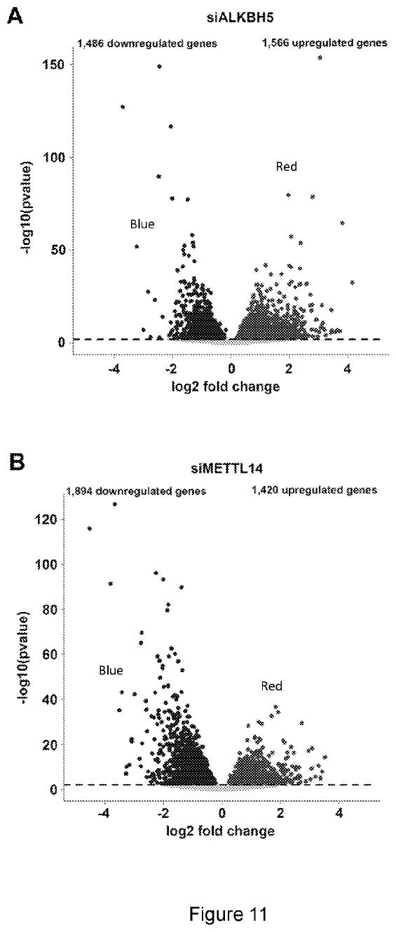

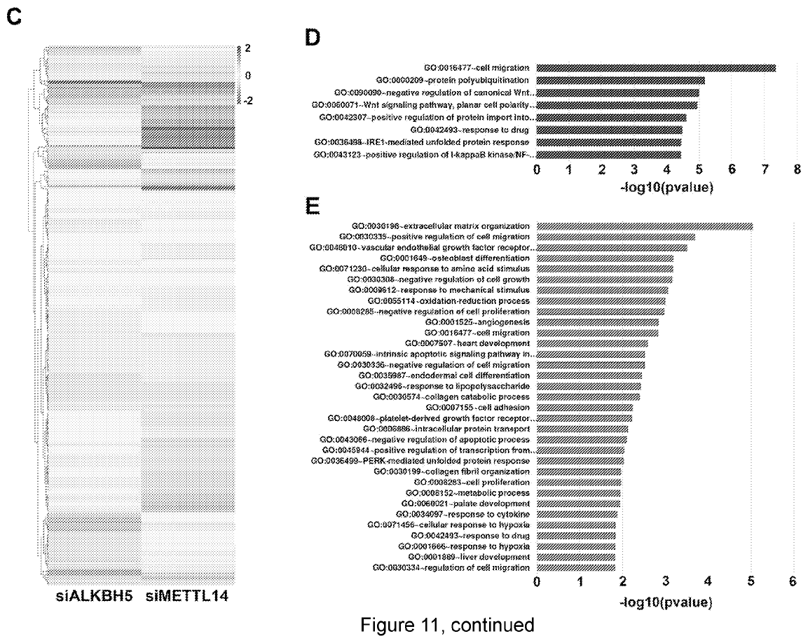

To determine the extent to which cellular m⁶A functions control genome-wide responses to dsDNA, total RNA isolated from primary normal human dermal fibroblasts (NHDFs) treated with non-silencing siRNA or siRNA specific for METTL14 or ALKBH5 and exposed to dsDNA for 6 and 12 h was analyzed by stranded mRNA sequencing (mRNA-seq). Following normalization and classification of differentially regulated genes (padj <0.01), 2,998 genes proved responsive to ALKBH5-depletion and 4,866 genes were responsive to METTL14-depletion after 12 h, compared to non-silencing siRNA-treated controls (FIG. 4A,B, FIG. 10). A similar number of differentially-expressed genes were observed in ALKBH5-depleted NHDFs treated with dsDNA for only 6 h (FIG. 11). However, the overall number of differentially-expressed genes in METTL14-depleted NHDFs exposed to dsDNA for 6 h was 30-40% less than that observed after 12 h (FIG. 11). The slower or delayed IFNB1 induction observed in METTL14-depleted cells (FIG. 11), which may extend to other genome-wide changes when 6 and 12 h time points are compared, likely accounts for this finding. It further suggests that the differential gene expression phenotypes, including those controlling IFNβ production, occur more rapidly upon m⁶A eraser-depletion than writer-depletion.

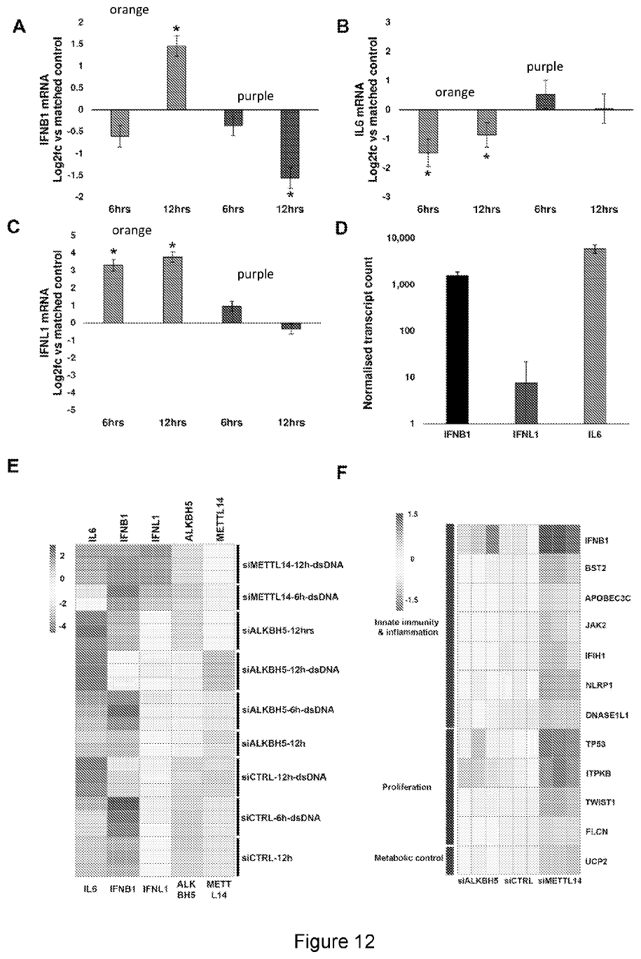

Compared to non-silencing siRNA-treated cultures, interfering with the m⁶A writer subunit METTL14 or the eraser ALKBH5 had a complex impact on gene expression in response to dsDNA (FIG. 4A,B). A greater number of genes (3419 genes) were uniquely regulated by METTL14-depletion compared to ALKBH5-depletion (1551 genes) after dsDNA treatment for 12 h (FIG. 4C). In addition, 984 genes were similarly co-regulated in the same direction by depleting either ALKBH5 or METTL14, while 463 genes were reciprocally regulated, including IFNB1 (FIG. 4C). Interferon and interferon stimulated genes (ISGs) figured prominently among those whose induction by dsDNA was impacted by interfering with ALKBH5 or METTL14 (FIG. 4D). Clustered heat maps revealed distinct signatures of IFNB1, IFNL1, and IL6 mRNA accumulation in NHDFs treated with non-silencing siRNA vs. siRNA specific for ALKBH5 or METTL14 in response to dsDNA (FIG. 12). Furthermore, 4% of a defined collection of 349 ISGs (Schoggins et al., 2011) together with notable genes controlling innate immunity and inflammation, proliferation, and metabolic control (FIG. 12) were upregulated by METTL14-depletion and downregulated by ALKBH5-depletion in response to dsDNA.

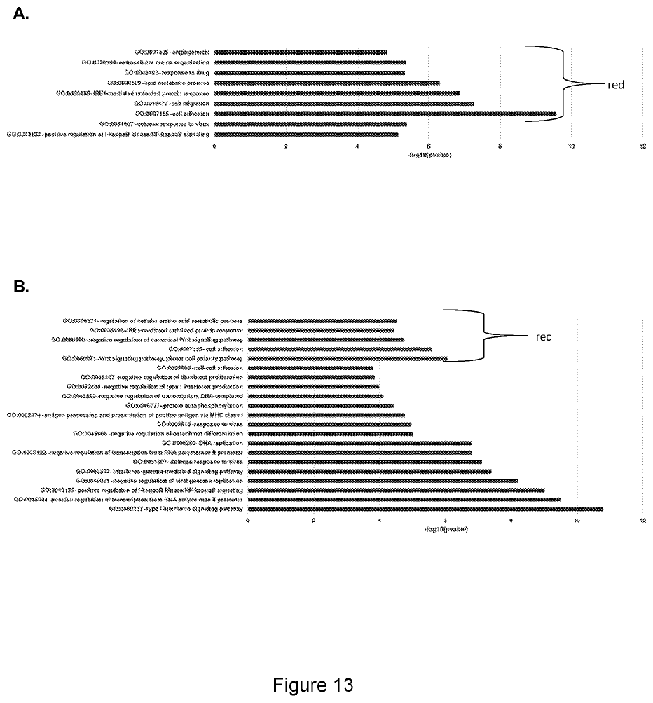

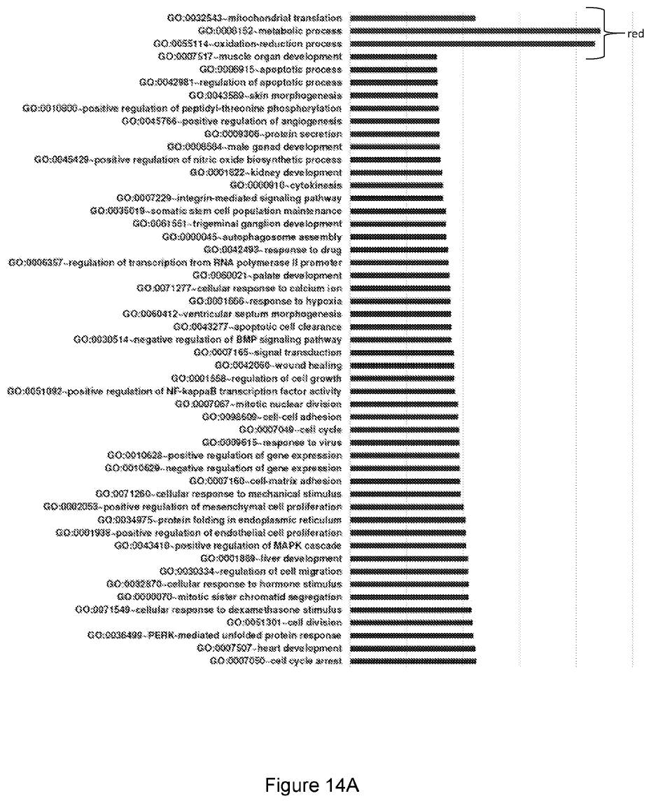

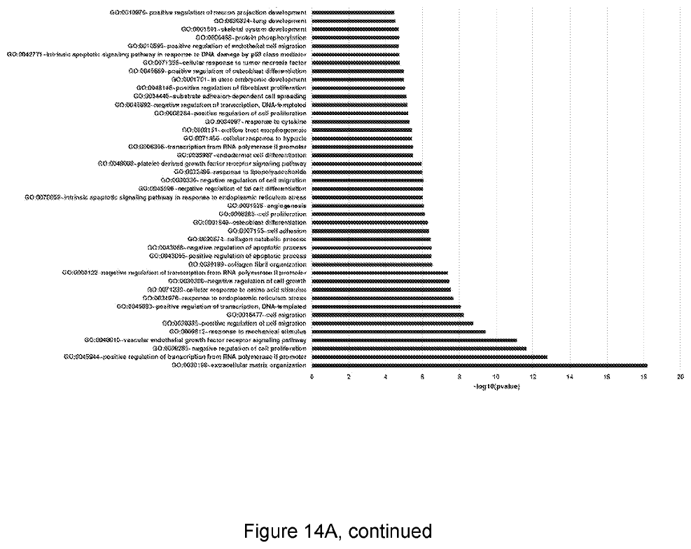

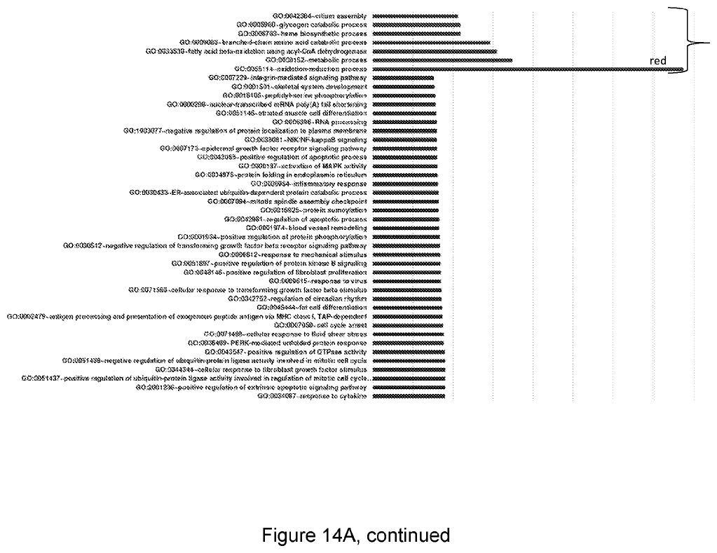

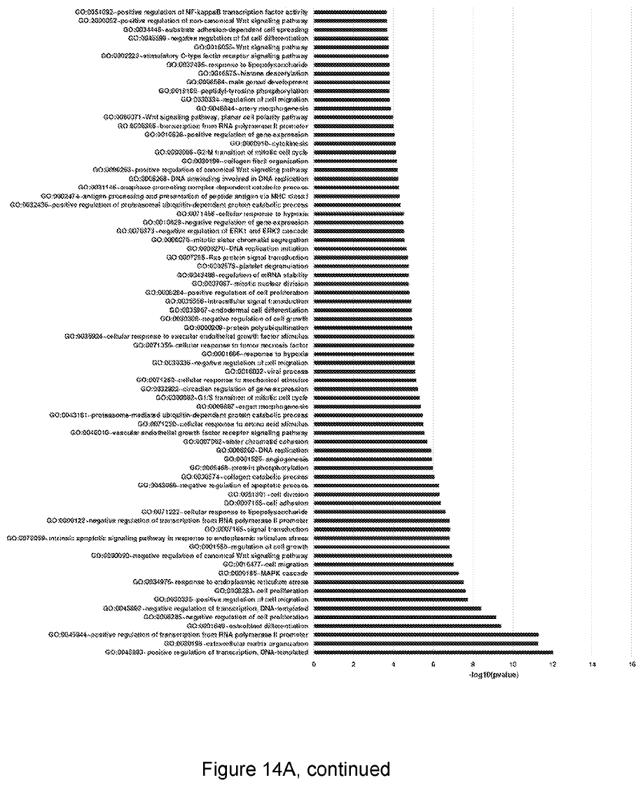

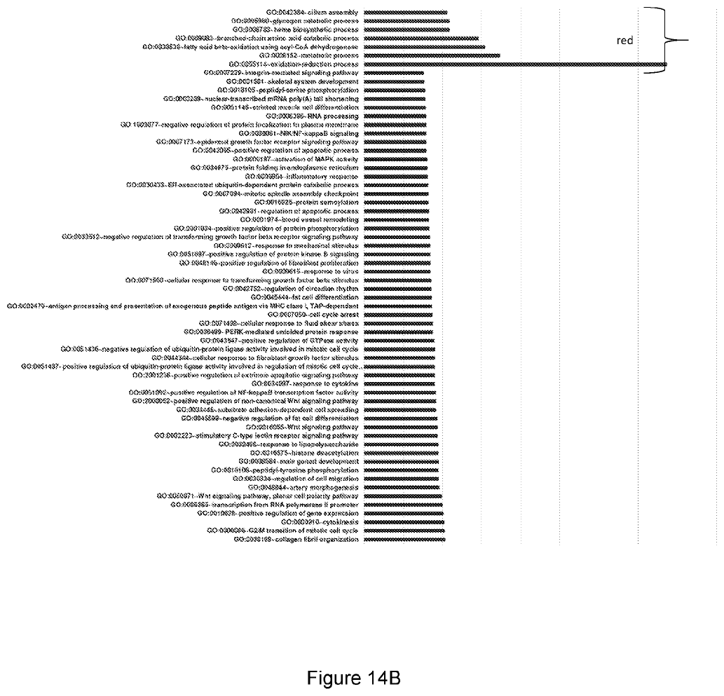

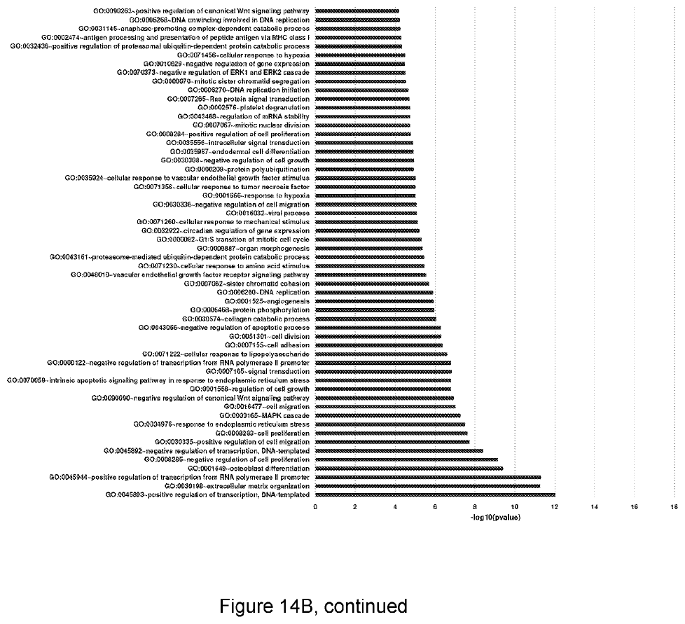

Top pathways enriched among genes differentially expressed upon ALKBH5-depletion in dsDNA treated NHDFs included those controlling signaling by interferon, Wnt, NF-κB TNF, TGFβ, LPS, many of which impact infection biology and immune responses (FIG. 4E). Notably, ALKBH5-depletion also impacted mRNA biogenesis, including pathways controlling RNA polymerase II transcription and mRNA stability (FIG. 4E). Pathway analysis of 1668 genes downregulated by ALKBH5-depletion include those controlling NF-κB activation, type I and type II IFN signaling, antigen processing and presentation, all of which have documented anti-viral roles (FIG. 13). By contrast, top pathways enriched among genes differentially expressed upon METTL14-depletion in dsDNA-treated NHDFs included those involved in oxidation-reduction, responses to amino acid stimulus, catabolic processes, hypoxia, aging, and ER stress (FIG. 4F). Pathway analysis of 2273 genes upregulated by METTL14-depletion in dsDNA-treated NHDFs include those involved in cilium assembly, catabolic processes and metabolic alterations (FIG. 14), some of which are associated with quiescence or differentiation, senescence and physiological stress (Favoro et al., 2012; Sanchez & Dynlacht, 2016; Takahashi et al, 2018). Analysis of pathways downregulated by METTL14-depletion include control of RNA polymerase II transcription, circadian regulation of gene expression, Wnt signaling, DNA replication, proliferation, responses to ER stress, LPS, TNF and hypoxia, and mRNA stability (FIG. 14). Overall this analysis shows that primary human fibroblasts express unique ALKBH5 or METTL14-dependent gene signatures in response to dsDNA. Significantly, while many of the genes differentially expressed following depletion of the ALKBH5 m⁶A demethylase impact cell intrinsic anti-viral immune responses, depleting the METTL14 m⁶A methyltransferase subunit impacts pathways involved in metabolic reprogramming, responses to physiological stress, and aging. These changes likely allow rapid metabolic adaptation to a changing environment.

### Example 5

IFNB1 mRNA Biogenesis and Decay are Regulated by m⁶A Modification Enzymes

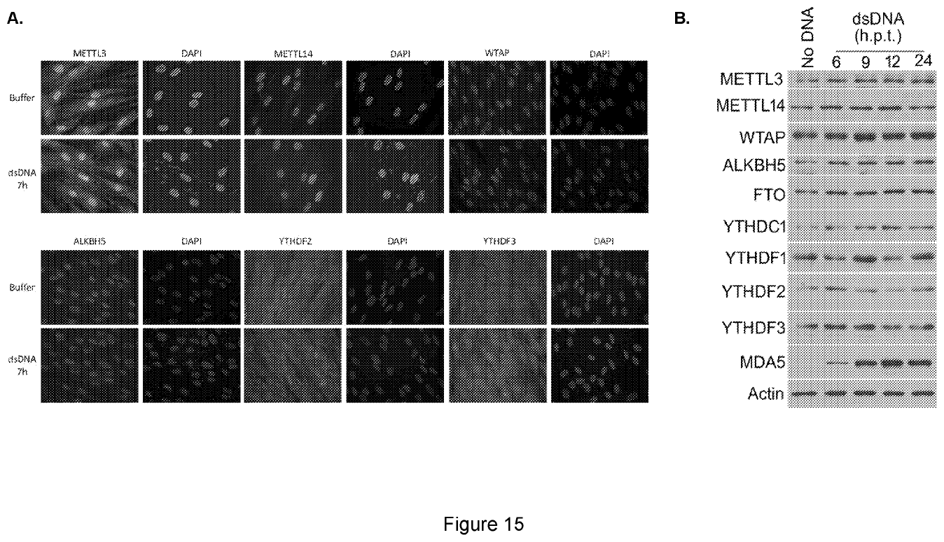

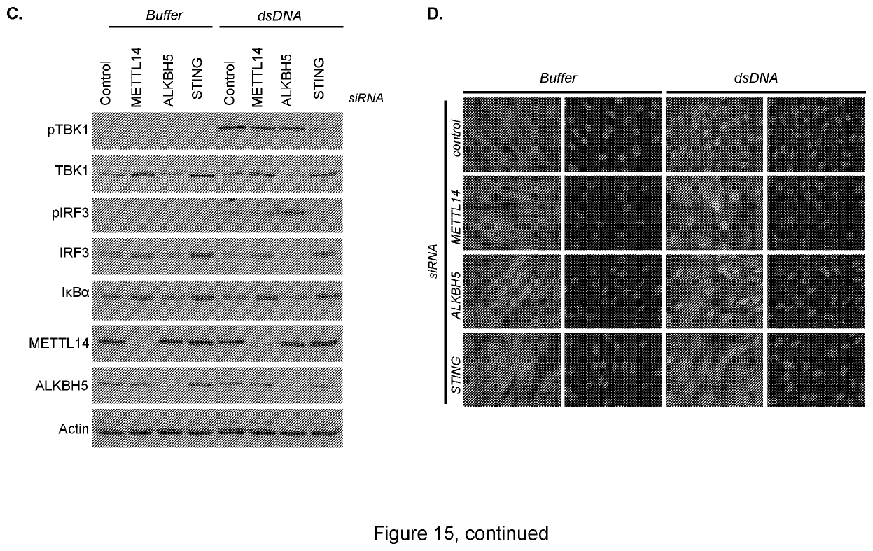

To more precisely understand how METTL14 and ALKBH5 control IFNB1 production, we sought to define the underlying regulatory mechanism(s). Treating NHDFs with dsDNA for 7 h did not significantly change the subcellular distribution or overall abundance of cellular m⁶A reader, writer, or eraser proteins (FIG. 15A,B). In addition, METTL14-depletion did not appreciably stimulate and ALKBH5-depletion did not detectably interfere with NF-κB and IRF3 signaling as measured by TBK or IRF3 phosphorylation, IκBα accumulation and IRF3 nuclear accumulation (FIG. 15C,D). This suggested that cellular m⁶A writers and erasers might be controlling IFNB1 mRNA metabolism, in particular mRNA biogenesis or decay of this unspliced cellular mRNA. To prevent secondary effects resulting from IFNβ cytokine signaling following IFNB1 mRNA production, we measured IFNB1 mRNA abundance in response to dsDNA in the presence and absence of JAK inhibitor (JAKi). FIG. 5A shows that compared to non-silencing siRNA-treated NHDFs, METTL14-depleted cells accumulate greater levels of IFNB1 mRNA from 9-13 h after dsDNA exposure. The kinetics of IFNB1 mRNA accumulation was faster in JAKi-treated cells. By contrast, reduced amounts of IFNB1 mRNA accumulated in ALKBH5-depleted cells (FIG. 5A).

To analyze how cellular m⁶A writers and erasers influenced mRNA biogenesis, nascent RNA metabolically pulse-labeled with ethyluridine (EU) for 30 min. was isolated from nuclei of dsDNA-treated cells to limit the contribution of cytoplasmic mRNA decay. FIG. 5B shows that EU-labeled IFNB1 mRNA levels in METTL14-depleted NHDFs increased compared to non-silencing siRNA-treated controls between 7-10 h after dsDNA treatment. In NHDFs treated with JAK inhibitor, a greater increase in EU-labeled IFNB1 mRNA in METTL14-depleted cultures compared to non-silencing siRNA-treated controls was observed earlier after only 7 h of dsDNA exposure. This indicated that the kinetics of IFNB1 mRNA production respond to IFN receptor signaling. Conversely, nuclei isolated from ALKBH5-depleted cultures contained less nascent EU-labeled IFNB1 mRNA compared to non-silencing siRNA-treated cultures (FIG. 5B).

To determine whether METTL14 or ALKBH5 also influenced IFNB1 mRNA decay, total EU pulse-labeled RNA was isolated from NHDFs treated with dsDNA and IFNB1 mRNA levels were measured by qRT-PCR. While decay of EU-labeled, nascent IFNB1 mRNA was indistinguishable in n.s. siRNA or ALKBH5-depleted cultures, EU-labeled IFNB1 mRNA persisted longer in METTL14-depleted cultures (FIG. 5C). By 11 h post-dsDNA treatment, less than 10% nascent IFNB1 mRNA remained in ns or ALKBH5-depleted cultures whereas nearly 30% was detected in METTL14-depleted NHDFs. Unexpectedly, the greater persistence of IFNB1 mRNA in METTL14-depleted NHDFs was dependent upon JAK signaling (FIG. 5C), consistent with an ISG selectively influencing the decay of hypo-m⁶A methylated IFNB1 mRNA population. Thus, the burst of IFNB1 mRNA biogenesis and decay in response to dsDNA is regulated by cellular functions that control m⁶A modification of mRNA. In addition, ALKBH5 and METTL14 had a greater impact on IFNB1 mRNA production than decay.

### Example 6

Materials & Methods

Cells, viruses and chemicals. Normal human dermal fibroblasts (NHDFs) (LONZA, CC-2509) were cultured in Dulbecco's modified Eagle's medium (DMEM, Corning 10-013-CV) supplemented with penicillin (100 U/ml), streptomycin (100 μg/ml) and 5% (v/v) fetal bovine serum (FBS; Invitrogen). Vero cells (ATCC) were propagated in DMEM supplemented with penicillin (100 U/ml), streptomycin (100 μg/ml) and 5% (v/v) calf serum (FBS; Invitrogen). BSC40 cells were maintained in DMEM supplemented with penicillin (100 U/ml), streptomycin (100 μg/ml) and 10% (v/v) FBS. HCMV AD169GFP was kindly provided by Dong Yu and was propagated in NHDFs as described (Bianco & Mohr, 2017). Vaccinia Virus (Western Reserve strain) was propagated in BSC40 cells as described (Burgess & Mohr, 2015). Wild-type HSV-1 (Patton strain) expressing an EGFP-Us11 fusion protein (Benboudjema et al. 2003) was propagated in Vero cells. PP242 (P0037) was purchased from Sigma and used at a concentration of 25 μM.

DNA transfections. DNA was transfected using TranslT-X2 (Mims MIR6000) according to the manufacturer's protocol. VACV 70-mer (dsDNA) oligonucleotides were described previously (Unterholzner et al. 2010). Annealing buffer (10 mM Tris-HCl, pH 7.5, 100 mM NaCl, and 1 mM EDTA) without nucleic acids was used as negative control.

m⁶A RNA immunoprecipitation (RIP). Following poly-A+ selection, purified polyadenylated RNA (˜1 μg) resuspended in IPP buffer (150 mM NaCl, 0.1% NP-40, 10 mM Tris-HCl, pH 7.5) was denatured at 70° C. for 2 min. and subsequently placed on ice. 25 μl of Protein-G magnetic beads (Life Technologies 10004D), washed and suspended in IPP buffer containing RNAse inhibitor (Invitrogen 10777019), were incubated with 1 μg of monoclonal anti-m⁶A antibody at RT for 15 min with end-over-end rotation. RNA was added to the antibody-bound beads and incubated for 2 h at 4° C. with end-over-end rotation. RNA-bound beads were washed twice in 200 μl IPP buffer, twice in low-salt IPP buffer (50 mM NaCl, 0.1% NP-40, 10 mM Tris-HCl, pH 7.5), twice in high-salt IPP buffer (500 mM NaCl, 0.1% NP-40, 10 mM Tris-HCl, pH 7.5), and twice again in 200 μl of IPP buffer and eluted in 30 μl RLT (QIAGEN 79216). To purify the RNA, 20 μl MyOne Silane Dynabeads (Life Technologies 37002D) was washed with 100 μl RLT, resuspended in 30 μl RLT, and added to the eluted RNA. After addition of 60 μl 100% ethanol, the beads were collected magnetically and the supernatant discarded. The beads were washed twice in 100 μl 70% ethanol, and the RNA eluted in 160 μl IPP buffer. A second round of IP was performed by incubating the RNA with protein-A magnetic beads (Life Technologies 10002D) coupled to anti-m⁶A antibody, followed by washes, elution from the protein-A beads and purification as described above. The final RNA elution from the MyOne Silane Dynabeads was performed in 16 μl H₂O followed immediately by cDNA synthesis.

Isolation of nuclear RNA. RNA from a nuclear fraction was isolated according to a protocol adapted from a published procedure (Wuarin & Schibler, 1994). Briefly, NHDFs were washed with PBS, scrapped int cold lysis buffer (10 mM Tris-HCl pH 7.5, 0.15% NP40, 150 mM NaCl) and incubated on ice for 5 min. The lysate was then transferred onto 2.5 volumes of a chilled sucrose cushion (ice-cold sucrose buffer [10 mM Tris-HCl pH 7.5, 150 mM NaCl, 24% sucrose]), and centrifuged at 13,000 rpm for 10 min at 4° C. The supernatant (cytoplasmic fraction) was collected for RNA extraction by TRIzol. The pellet was resuspended in cytoplasmic lysis buffer without NP40 and passed through a fresh sucrose cushion for a second time. The washed nuclear fraction was then dissolved in TRIzol for RNA extraction.

Analysis of nascent RNA synthesis and decay. Transcription and stability of newly synthesized RNA were analysed using the Click-iT® Nascent RNA Capture Kit (Life Technologies C10365). To measure RNA decay, siRNA-treated NHDFs were stimulated with dsDNA. After 3 h, 0.2 mM Ethyluridine (EU) was added. Following a 2 h incubation with EU, the cells were washed with PBS and EU-free medium was added. Cells were harvested and RNA isolated with TRIzol at the indicated time points after the addition of EU-free media. The EU-labeled RNAs were biotinylated and captured by using the Click-iT® Nascent RNA Capture Kit, according to the manufacturer's instructions. To measure EU incorporation into newly synthesized nuclear RNA, siRNA-treated NHDFs stimulated with dsDNA were exposed to a 30 min EU pulse at the indicated times post-DNA addition. Following EU exposure, cells were washed with PBS, nuclear RNA was isolated as described in the preceding paragraph, and processed as described above.

Library preparation and sequencing. Illumina TruSeq stranded RNA libraries were prepared from poly(A)-selected total RNA (500 ng, RIN score 10) by staff at the New York University Genome Technology Center and sequenced across two PE150 Cycle lanes of a HiSeq 4000, yielding between 20,227,308-28,768,351 paired-end reads per sample (Table 1).

TABLE 1

| | Total | Pseudoaligned | % Pseudoaligned | SRA | |
|---|---|---|---|---|---|
| Sample ID | reads | reads | reads | accession | Study ID |
| siALK-12h-1 | 27,577,975 | 19,387,112 | 70.30 | SRR7049604 | SRP141411 |
| siALK-12h-2 | 23,119,676 | 14,714,334 | 63.64 | SRR7049603 | SRP141411 |
| siALK-12h-3 | 26,914,918 | 18,226,817 | 67.72 | SRR7049606 | SRP141411 |
| siALK-12h-dsDNA-1 | 25,423,948 | 16,182,307 | 63.65 | SRR7049605 | SRP141411 |
| siALK-12h-dsDNA-2 | 23,796,444 | 16,259,094 | 68.33 | SRR7049608 | SRP141411 |
| siALK-12h-dsDNA-3 | 26,040,459 | 17,643,600 | 67.75 | SRR7049607 | SRP141411 |
| siALK-6h-dsDNA-1 | 27,113,209 | 18,473,963 | 68.14 | SRR7049600 | SRP141411 |
| siALK-6h-dsDNA-2 | 26,632,328 | 17,851,911 | 67.03 | SRR7049599 | SRP141411 |
| siALK-6h-dsDNA-3 | 27,310,965 | 18,645,613 | 68.27 | SRR7049595 | SRP141411 |
| siCTRL-12h-1 | 27,841,502 | 19,164,375 | 68.83 | SRR7049612 | SRP141411 |
| siCTRL-12h-2 | 24,816,513 | 17,167,924 | 69.18 | SRR7049611 | SRP141411 |
| siCTRL-12h-3 | 26,864,295 | 18,714,077 | 69.66 | SRR7049610 | SRP141411 |
| siCTRL-12h-dsDNA-1 | 25,398,238 | 17,262,131 | 67.97 | SRR7049609 | SRP141411 |
| siCTRL-12h-dsDNA-2 | 24,975,861 | 17,013,712 | 68.12 | SRR7049616 | SRP141411 |
| siCTRL-12h-dsDNA-3 | 25,803,024 | 17,809,057 | 69.02 | SRR7049615 | SRP141411 |
| siCTRL-6h-dsDNA-1 | 27,382,992 | 18,953,034 | 69.21 | SRR7049614 | SRP141411 |
| siCTRL-6h-dsDNA-2 | 25,875,984 | 17,620,743 | 68.10 | SRR7049613 | SRP141411 |
| siCTRL-6h-dsDNA-3 | 26,161,781 | 17,735,955 | 67.79 | SRR7049617 | SRP141411 |
| siL14-12h-1 | 24,022,747 | 16,031,935 | 66.74 | SRR7049596 | SRP141411 |
| siL14-12h-2 | 20,227,308 | 13,331,114 | 65.91 | SRR7049597 | SRP141411 |
| siL14-12h-3 | 24,297,328 | 16,805,940 | 69.17 | SRR7049598 | SRP141411 |
| siL14-12h-dsDNA-1 | 23,376,433 | 15,874,448 | 67.91 | SRR7049591 | SRP141411 |
| siL14-12h-dsDNA-2 | 22,322,121 | 14,883,534 | 66.68 | SRR7049592 | SRP141411 |
| siL14-12h-dsDNA-3 | 26,387,134 | 18,140,154 | 68.75 | SRR7049593 | SRP141411 |
| siL14-6h-dsDNA-1 | 23,244,321 | 15,522,639 | 66.78 | SRR7049594 | SRP141411 |
| siL14-6h-dsDNA-2 | 28,768,351 | 5,865,627 | 20.39 | SRR7049589 | SRP141411 |
| siL14-6h-dsDNA-3 | 24,024,280 | 16,500,107 | 68.68 | SRR7049590 | SRP141411 |

For m⁶A RNA sequencing, RNA fragments with m⁶A-modifications were captured by RIP as described in the preceding section except that a polyclonal m⁶A antibody was used (Dominissini et al, 2013). m⁶A-Seq IP and input libraries were prepared from 5-10 ng of RNA using the NEBNext® Ultra™ II RNA Library Prep (New England Biolabs) following the FFPE entry protocol. Twelve libraries were multiplexed and sequenced on an Illumina NextSeq 500 using a single 75 cycle high output kit v2 (single end read mode), yielding 27,000,000-42,000,000 single-end reads per sample (Table 2). Analysis of m⁶A sequencing is described in the supplementary methods.

TABLE 2

| | | | % | | % unique- | |
|---|---|---|---|---|---|---|
| | | Mapping | mapping | Unique-mapping | mapping | SRA |
| Sample | Total reads | reads | reads | reads | reads | accession |
| bufINPUT1 | 39,446,113 | 11,636,466 | 79.95 | 9,596,115 | 82.47 | SRR7992458 |
| bufINPUT2 | 36,598,934 | 10,534,157 | 79.04 | 8,665,662 | 82.26 | SRR7992461 |
| bufINPUT3 | 36,955,536 | 11,321,597 | 79.69 | 9,450,692 | 83.47 | SRR7992460 |
| bufIP1 | 38,452,765 | 12,259,651 | 82.98 | 10,562,969 | 86.16 | SRR7992455 |
| bufIP2 | 49,975,935 | 14,733,666 | 83.28 | 12,676,820 | 86.04 | SRR7992454 |
| bufIP3 | 27,130,961 | 9,935,800 | 83.72 | 8,511,744 | 85.67 | SRR7992459 |
| dsINPUT1 | 42,503,116 | 11,040,090 | 72.28 | 9,329,018 | 84.5 | SRR7992450 |
| dsINPUT2 | 32,864,139 | 8,988,320 | 73.93 | 7,566,020 | 84.18 | SRR7992457 |
| dsINPUT3 | 42,746,912 | 11,050,076 | 72.69 | 9,393,032 | 85 | SRR7992456 |
| dsIP1 | 33,580,979 | 10,449,366 | 79.76 | 9,078,676 | 86.88 | SRR7992453 |
| dsIP2 | 48,038,886 | 13,379,307 | 78.17 | 11,636,545 | 86.97 | SRR7992452 |
| dsIP3 | 40,230,672 | 11,960,350 | 80.68 | 10,471,851 | 87.55 | SRR7992451 |

RNASeq analysis. Sequence reads were pseudoaligned against a Homo sapiens transcriptome database comprising the latest cDNA and ncRNA databases from Ensembl using Kallisto (Bray et al., 2016) under default parameters. Raw transcript counts were parsed to generate raw gene counts and analysed using the DESeq2 v1.18.1 (Love et al., 2014) package in R. Two samples (siAlk-12 h-2 and siL14-6 h-dsDNA-2) were excluded from analyses based on PCA clustering and low total read counts, respectively (FIG. 8). Pairwise contrasts were performed between sample sets for each condition (12 hrs, no dsDNA/6 hrs+dsDNA/12 hrs+dsDNA) and filtered to retain only differentially expressed genes with adjusted p-values (padj)<0.01. Pathway analyses for resulting gene lists were performed using the ‘Functional Annotation Tool’ hosted by the David Bioinformatics Research 6.8 platform (Huang et al, 2009a,b).

Data availability. All sequencing data generated during this study are available from the sequence read archive (SRA) under the BioProject ID PRJNA451188.

References for the foregoing description.
1. Aguilo F, Zhang F, Sancho A, Fidalgo M, Di Cecilia S, Vashisht A, Lee D F, Chen C H, Rengasamy M, Andino B, et al. 2015. Coordination of m⁶A mRNA methylation and gene transcription by ZFP217 regulates pluripotency and reprogramming. Cell Stem Cell 17: 689-704.
2. Benboudjema L, Mulvey M, Gao Y, Pimplikar S W, Mohr I. 2003. Association of the herpes simplex virus type 1 Us11 gene product with the cellular kinesin light-chain related protein PAT1 results in the redistribution of both polypeptides. J. Virol. 77: 9192-203.
3. Bianco C, Mohr I. 2017. Restriction of Human Cytomegalovirus Replication by ISG15, a Host Effector Regulated by cGAS-STING Double-Stranded-DNA Sensing. J. Virol. 91: pii: e02483-16.
4. Boeckh M, Geballe A P. 2011. Cytomegalovirus: pathogen, paradigm, and puzzle. J Clin Invest 121: 1673-1680.
5. Britt W. 2008. Manifestations of human cytomegalovirus infection: proposed mechanisms of acute and chronic disease, p 417-470. In Shenk T E, Stinski M F. (ed), Human cytomegalovirus. Springer, Berlin, Germany.
6. Bray N L, Pimentel H, Melsted P, Pachter L. 2016. Near-optimal probabilistic RNA-seq quantification. Nat. Biotechnol. https://doi.org/10.1038/nbt.3519.
7. Brocard M, Ruggieri A, Locker N. 2017. m⁶A RNA methylation, a new hallmark in virus-host interactions. J. Gen. Virol. 98: 2207-2214.
8. Burgess, H M, Mohr I. 2015. Cellular 5′-3′ mRNA exonuclease Xrn1 controls double-stranded RNA accumulation and anti-viral responses. Cell Host Microbe 17: 332-344.
9. Cannon M J, Schmid D S, Hyde T B. 2010. Review of cytomegalovirus seroprevalence and demographic characteristics associated with infection. Rev Med Virol 20: 202-213.
10. Coots R A, Liu X M, Mao Y, Dong L, Zhou J, Wan J, Zhang X, Qian S B. 2017. m⁶A Facilitates eIF4F-Independent mRNA Translation. Mol Cell. 68: 504-514.e7.
11. Chen C A, Shyu A B. 2017. Decay Emerging Themes in Regulation of Global mRNA Turnover in cis. Trends Biochem Sci. 42: 16-27.
12. Chen Q, Sun L, Chen Z J. 2016. Regulation and function of the cGAS-STING pathway of cytosolic DNA sensing. Nat Immunol. 17: 1142-1149.
13. Coppé J-P, Patil, C K, Rodier F, Sun Y, Munoz D P, Goldastein J, Nelson P S, Desprez P-Y, Campisi J. 2008. Senescence-Associated Secretory Phenotypes Reveal Cell-Nonautonomous Functions of Oncogenic RAS and the p53 Tumor Suppressor. PLoS Biol. 6: e301.
14. Courtney D G, Kennedy E M, Dumm R E, Bogerd H P, Tsai K, Heaton N S, Cullen B R [symbol] 2017. Epitranscriptomic Enhancement of Influenza A Virus Gene Expression and Replication. Cell Host & Microbe 22: 377-386.
15. Crowl J T, Gray E E, Pestal K, Volkman H E, Stetson D B. 2017. Intracellular Nucleic Acid Detection in Autoimmunity. Ann. Rev. Immunol. 35: 313-336.
16. Dominissini D, Moshitch-Moshkovitz S, Salmon-Divon M, Amariglio N, Rechavi G. 2013. Transcriptome-wide mapping of N(6)-methyladenosine by m(6)A-seq based on immuno-capturing and massively parallel sequencing. Nat Protoc. 8: 176-189.
17. Dominissini D, Moshitch-Moshkovitz S, Schwartz S, Salmon-Divon M, Ungar L, Osenberg S, Cesarkas K, Jacob-Hirsch J, Amariglio N, Kupiec M, et al. 2012. Topology of the human and mouse m6A RNA methylomes revealed by m6A-seq. Nature 485: 201-206.
18. Du H, Zhao Y, He J, Zhang Y, Xi H, Liu M, Ma J, Wu L. 2016. YTHDF2 destabilizes m6A-containing RNA through direct recruitment of the CCR4-NOT deadenylase complex. Nat. Commun. 7: 12626.
19. Edupuganti R R, Geiger S, Lindeboom R G H, Shi H, Hsu P J, Lu Z, Wang S Y, Baltissen M P A, Jansen PWTC, Rossa M, et al. 2017. N6-methyladenosine (m6A) recruits and repels proteins to regulate mRNA homeostasis. Nat Struct Mol Biol. 24: 870-878.
20. Fustin J.-M. et al. 2013. RNA-methylation-dependent RNA processing controls the speed of the circadian clock. Cell 155: 793-806.
21. Favaro E, Bensaad K, Chong M G, Tennant D A, Ferguson D J P, Snell C, Steers G, Turley H, Li J-L, Gunther U L, et al. (2012). Glucose Utilization via Glycogen Phosphorylase Sustains Proliferation and Prevents Premature Senescence in Cancer Cells. Cell Metab. 9. 16: 751-764.
22. Gao D, Li T, Li X D, Chen X, Li Q Z, Wight-Carter M, Chen Z J. 2015. Activation of cyclic GMP-AMP synthase by self-DNA causes autoimmune diseases. Proc Natl Acad Sci USA 112: E5699-705.
23. Gokhale N S, McIntyre A B, McFadden M J, Roder A E, Kennedy E M, Gandara J A, et al. 2016. N6-Methyladenosine in Flaviviridae Viral RNA Genomes Regulates Infection. Cell Host Microbe 20: 654-665.
24. Gonzales-van Horn S R, Sarnow P. 2017. Making the Mark: The Role of Adenosine Modifications in the Life Cycle of RNA Viruses. Cell Host Microbe. 21: 661-669.
25. Haussmann I U, Bodi Z, Sanchez-Moran E, Mongan N P, Archer N, Fray R G, Soller M. 2016. m6A potentiates Sxl alternative pre-mRNA splicing for robust Drosophila sex determination. Nature. 540: 301-304.
26. Herdy B K T, Vladimer G. I, Tan, C S H, Stukalov A, Trefzer C Bigenzahn J. 2015. The RNA-binding protein HuR/ELAVL1 regulates IFN-β mRNA abundance and the type I IFN response. Eur. Immunol. 45: 1500-1511.
27. Hesser C R, Karijolich J, Dominissini D, He C, Glaunsinger B A. 2018. N6-methyl-adenosine modification and the YTHDF2 reader protein play cell type specific roles in lytic viral gene expression during Kaposi's sarcoma-associated herpesvirus infection. PLoS Pathog. 14, e1006995.
28. Huang D W, Sherman B T, Lempicki R A. 2009a. Bioinformatics enrichment tools: Paths toward the comprehensive functional analysis of large gene lists. Nuc. Acids Res. https://doi.org/10.1093/nar/gkn923.
29. Huang D W, Sherman B T, Lempicki R A. 2009b. Systematic and integrative analysis of large gene lists using DAVID bioinformatics resources. Nat. Protoc. https://doi.org/10.1038/nprot.2008.211.
30. Ishikawa H, Ma Z, Barber G N. 2009. STING regulates intracellular DNA-mediated, type I interferon-dependent innate immunity. Nature. 461:, 788-792.
31. Jia G, Fu Y, Zhao X, Dai Q, Zheng G, Yang Y, Yi C, Lindahl T, Pan T, Yang Y G, He C. 2011. N6-methyladenosine in nuclear RNA is a major substrate of the obesity-associated FTO. Nat. Chem. Biol. 7: 885-887.
32. Ke S, Pandya-Jones, S Y, Fak J J, Vågbø C B, Geula S, Hanna J H, Black D L, Darnell J E Jr, Darnell R B. 2017. m⁶A mRNA modifications are deposited in nascent pre-mRNA and are not required for splicing but do specify cytoplasmic turnover. Genes Dev. 31: 990-1006.
33. Kennedy E M, Bogerd H P, Kornepati A V R, Kang D, Ghoshal D, Marshall J B, Poling, B C, Tsai K, Gokhale N S, Homer S M, Cullen B R. 2016. Post-transcriptional m(6)A Editing of HIV-1 mRNAs Enhances Viral Gene Expression. Cell Host Microbe 19: 675-685.
34. Lavi S, Shatkin A J. 1975. Methylated simian virus 40-specific RNA from nuclei and cytoplasm of infected BSC-1 cells. Proc Natl Acad Sci USA 72: 2012-2016.
35. Li T, Chen Z J. 2018. The cGAS-cGAMP-STING pathway connects DNA damage to inflammation, senescence, and cancer. J Exp Med. April 5. pii: jem.20180139.
36. Li L J, Fan Y G, Leng R X, Pan H F, Ye D Q. 2018. Potential link between m6A modification and systemic lupus erythematosus. Mol Immunol. 93: 55-63.
37. Lichinchi G, Gao S, Saletore Y, Gonzalez G M, Bansal V, Wang Y, Mason C E, Rana T M. 2016a. Dynamics of the human and viral m(6)A RNA methylomes during HIV-1 infection of T cells. Nat. Microbiol. 1: 16011.
38. Lichinchi G, Zhao B S, Wu Y, Lu Z, Qin Y, He C, Rana T M. 2016b. Dynamics of Human and Viral RNA Methylation during Zika Virus Infection. Cell Host Microbe 20: 666-673.
39. Linder B, Grozhik A V, Olarerin-George A O, Meydan C, Mason C E, Jaffrey S R. 2015. Single-nucleotide-resolution mapping of m6A and m6Am throughout the transcriptome. Nat Methods. 12: 767-772.
40. Liu N, Dai Q, Zheng G, He C, Parisen M, Pan T. 2015. N6-methyl-adenosine-dependent RNA structural switches regulate RNA-protein interactions. Nature 518: 560-564.
41. Liu N, Zhou K I, Parisien M, Dai Q, Diachenko L, Pan T. 2017. N6-methyladenosine alters RNA structure to regulate binding of a low-complexity protein. Nucleic Acids Res. 45, 6051-6063.
42. Liu J, Yue Y, Han D, Wang X, Fu Y, Zhang L, Jia G, Yu M, Lu Z, Deng X, Dai Q, Chen W, He C. (2014). A METTL3-METTL14 complex mediates mammalian nuclear RNA N6-adenosine methylation. Nat. Chem. Biol. 10: 93-95.
43. Ljungman P, Hakki M, Boeckh M. 2010. Cytomegalovirus in hematopoietic stem cell transplant recipients. Infect Dis. Clin. North Am. 24: 319-337.
44. Love M I, Huber W, Anders S. 2014. Moderated estimation of fold change and dispersion for RNA-seq data with DESeq2. Genome iol. haps://doi org/10.1186/s13059-014-0550-8.
45. Manicklal S, Emery V C, Lazzarotto T, Boppana S B, Gupta R K. 2013. The “silent” global burden of congenital cytomegalovirus. Clin. Microbiol. Rev. 26: 86-102.
46. Mauer J, Luo X, Blanjoie A, Jiao X, Grozhik A V, Patil D P, Linder B, Pickering B F, Vasseur J J, Chen Q, Gross S S, Elemento O, Debart F, Kiledjian M, Jaffrey S R. 2017. Reversible methylation of m6Am in the 5=cap controls mRNA stability. Nature 541: 371-375.
47. McKinney C, Perez C, Mohr I. 2012. Poly(A) binding protein abundance regulates eukaryotic translation initiation factor 4F assembly in human cytomegalovirus-infected cells. Proc Natl Acad Sci USA. 109: 5627-5632.
48. McKinney C, Zavadil J, Bianco C, Shiflett L, Brown S, Mohr I. 2014. GlobalReprogramming of the Cellular Translational Landscape Facilitates Cytomegalovirus Replication. Cell Rep. 6: 9-17.
49. Mer Merkestein M, Laber S, McMurray F, Andrew D, Sachse G, Sanderson J, Li M, Usher S, Sellayah D, Ashcroft F M, Cox R D. 2015. FTO influences adipogenesis by regulating mitotic clonal expansion. Nat. Commun. 6: 6792.
50. Meyer K D, Patil D P, Zhou J, Zinoviev A, Skabkin M A, Elemento O, Pestova T V, Qian S B, Jaffrey S R. 2015. 5′ UTR m6A promotes cap-independent translation. Cell 163: 999-1010.
51. Meyer K D, Saletore Y, Zumbo P, Elemento O, Mason C E, Jaffrey S R. 2012. Comprehensive analysis of mRNA methylation reveals enrichment in 30 UTRs and near stop codons. Cell 149: 1635-1646.
52. Moss B, Gershowitz A, Stringer J R, Holland L E, Wagner E K. 1977. 5′-Terminal and internal methylated nucleosides in herpes simplex virus type 1 mRNA. J. Virol. 23: 234-239.
53. Patil D P, Pickering B F, Jaffrey S R. 2018. Reading m6A in the Transcriptome: m6A-Binding Proteins. Trends Cell Biol. 28: 113-127.
54. Razonable R R, Humar A., 2013. AST Infectious Diseases Community of Practice. Cytomegalovirus in solid organ transplantation. Am. J. Transplant 13: 93-106.
55. Roost C. et al. 2015. Structure and thermodynamics of N6-methyladenosine in RNA: a spring-loaded base modification. J. Am. Chem. Soc. 137: 2107-2115.
56. Rosa-Mercado N A, Withers J B, Steitz J A. 2017. Settling the m6A debate: methylation of mature mRNA is not dynamic but accelerates turnover. Genes Dev. 31: 957-958.
57. Roundtree I A, Evans M E, Pan T, He C. 2017. Dynamic RNA Modifications in Gene Expression Regulation. Cell 169: 1187-1200.
58. Sanchez I, Dynlacht B D 2016. Cilium assembly and disassembly. Nat Cell Biol. 18: 711-717.
59. Schoggins J W, Wilson S J, Panis M, Murphy M Y, Jones C T, Bieniasz P, Rice C M. 2011. A diverse range of gene products are effectors of the type I interferon antiviral response. Nature. 472: 481-485.
60. Sommer S, Salditt-Georgieff M, Bachenheimer S, Darnell J E, Furuichi Y, Morgan M, et al. 1976. The methylation of adenovirus-specific nuclear and cytoplasmic RNA. Nuc. Acids Res. 3: 749-765.
61. Takahashi A, Loo T M, Okada R, Kamachi F, Watanabe Y, Wakita M, Watanabe S, Kawamoto S, Miyata K, Barber G N, Ohtani N, Hara E. 2018. Downregulation of cytoplasmic DNases is implicated in cytoplasmic DNA accumulation and SASP in senescent cells. Nat Commun. 9: 1249.
62. Takiuichi, O. 2015. HuR keeps interferon-β mRNA stable. Eur. J. Immunol. 45: 1296-1299.
63. Tan, B., Liu H, Zhang S, da Silva S R, Zhang L, Meng J, Cui X, Yuan H, Sorel O, Zhang S W, et al., 2018. Viral and cellular N6-methyladenosine and N6,2′-O-dimethyl-adenosine epitranscriptomes in the KSHV life cycle. Nat Microbiol. 3: 108-120.
64. Tirosh O, Cohen Y, Shitrit A, Shani O, Le-Trilling V T K, Trilling M, Friedlander G, Tanenbaum M, Stern-Ginossar N. (2015). The transcription and translation landscapes during human cytomegalovirus infection reveal novel host-pathogen interactions. PLoS Pathog 11: e1005288.
65. Tirumuru N, Zhao B S, Lu W, Lu Z, He C, Wu L. 2016. N(6)-methyl-adenosine of HIV-1 RNA regulates viral infection and HIV-1 Gag protein expression. eLife 5: e15528.
66. Tsai K, Courtney D G, Cullen B R. 2018. Addition of m6A to SV40 late mRNAs enhances viral structural gene expression and replication. PLoS Pathog. 14: e1006919.
67. Unterholzner et al. 2010. IFI16 is an innate immune sensor for intracellular DNA. Nat. Immunol. 11: 997-1004.
68. Vu L P, Pickering B F, Cheng Y, Zaccara S, Nguyen D, Minuesa G, Chou T, Chow A, Saletore Y, MacKay M, et al. 2017. The N6-methyladenosine (m6A)-forming enzyme METTL3 controls myeloid differentiation of normal hematopoietic and leukemia cells. Nat Med. 23: 1369-1376.
69. Wang X, Lu Z, Gomez A, Hon G C, Yue Y, Han D, Fu Y, Parisien M, Dai Q, Jia G, et al. 2014) N6-methyladenosine-dependent regulation of messenger RNA stability. Nature. 505: 117-20.
70. Wuarin J, Schibler U. 1994. Physical isolation of nascent RNA chains transcribed by RNA polymerase II: evidence for cotranscriptional splicing. Mol. Cell. Biol. 14: 7219-7225.
71. Wen J, Lv R, Ma H, Shen H, He C, Wang J, Jiao F, Liu H, Yang P, Tan L, et al., 2018. Zc3 h13 Regulates Nuclear RNA m⁶A Methylation and Mouse Embryonic Stem Cell Self-Renewal. Mol Cell. 69: 1028-1038.
72. Wojtas M N, Pandey R R, Mendel M, Homolka D, Sachidanandam R, Pillai R S. 2017. Regulation of m6A Transcripts by the 3′→5′ RNA Helicase YTHDC2 Is Essential for a Successful Meiotic Program in the Mammalian Germline. Mol Cell. 68: 374-387.
73. Yan N. 2017. Immune Diseases Associated with TREX1 and STING Dysfunction. J. Interferon Cytokine Res. 37:198-206.
74. Yue Y, Liu J, He C. 2015. RNA N6-methyladenosine methylation in post-transcriptional gene expression regulation. Genes Dev. 29: 1343-1355.
75. Zheng, G. Q. et al. 2013. ALKBH5 is a mammalian RNA demethylase that impacts RNA metabolism and mouse fertility. Mol. Cell 49:18-29.
76. Zheng Q, Hou J, Zhou Y, Li Z, Cao X. 2017. The RNA helicase DDX46 inhibits innate immunity by entrapping m6A-demethylated antiviral transcripts in the nucleus. Nat. Immunol. 18: 1094-1103.
77. Zhou J, Wan J, Gao X, Zhang X, Jaffrey S R, Qian S B. 2015. Dynamic m(6)A mRNA methylation directs translational control of heat shock response. Nature. 526: 591-594
78. Zhou J, Wan J, Shu X E, Mao Y, Liu X M, Yuan X, Zhang X, Hess M E, Bruning J C, Qian S B. 2018. N⁶Methyladenosine Guides mRNA Alternative Translation during Integrated Stress Response. Mol Cell 69: 636-647.
79. Zhu H, Cong J P, Shenk T. 1997. Use of differential display analysis to assess the effect of human cytomegalovirus infection on the accumulation of cellular RNAs: induction of interferon-responsive RNAs. Proc. Natl. Acad. Sci. USA. 94 13985-13990.

### Example 7

Supplemental Materials & Methods

Immunoblotting and Antibodies. Total Cellular Protein was Collected by Lysis in sample buffer (62.5 mM Tris-HCl pH 6.8, 2% SDS, 10% glycerol, 0.7M β-mercaptoethanol) followed by boiling for 3 min. Lysates were fractionated by SDS-PAGE and analyzed by immunoblotting using the following antibodies: anti-ALKBH5 (Sigma SAB1407587), anti-FTO (Abcam ab124892), mouse anti-m⁶A monoclonal (Synaptic Systems 202111), rabbit anti-m6A polyclonal (Synaptic Systems 202003), anti-METTL14 (Sigma HPA038002), anti-METTL3 (Proteintech 15073-1 AP), anti-WTAP (Proteintech 60188-1-Ig), anti-YTHDF1 (Proteintech 17479-1-AP), anti-YTHDF2 (Proteintech 24744-1-AP), anti-YTHDF3 (Proteintech 25537-1-AP), anti-YTHDC1 (Abeam ab122340), anti-STAT1 (Cell Signaling 9172), anti-STAT1 Y701 (Cell Signaling 7649), anti-MIDAS (Proteintech 21775-1-AP), anti-pTBK1 S172 (Cell Signaling 5483), anti-TBK1 (Cell Signaling 3504), anti-pIRF3 S396 (Cell Signaling 4947), anti-IRF3 (Proteintech 11312-1-AP), anti-11(13a (Proteintech 10268-1-AP), anti-4E-BP1 (Bethyl Laboratories A300-501A), anti-actin (Cell Signaling 3700), anti-actin (Cell Signaling 3700), anti-GAPDH (Cell Signaling 2118); anti-pp28 (Virusys CA004-100), anti-UL44 (Virusys CA006), and anti-IE1/IE2 (Millipore MAB810). Primary antibodies were detected using either anti-mouse IgG HRP (GE Healthcare NA93 IV) or anti-rabbit IgG HRP (GE Healthcare NA934V) secondary antibodies and visualized by chemiluminescent detection.

RNA interference. siRNAs (20 nM) were transfected using RNAimax (Invitrogen 13778075) according to the manufacturer's instructions. METTL3-specific siRNA [5′-CUGCAAGUAUGUUCACUAUGA-3′, (SEQ ID NO:23) Liu et al., 2014] was synthesized by Sigma. The following siRNAs were purchased from Sigma: METTL14 (SASI_Hs01_00179440), ALKBH5 (SASI_Hs01_00013942), FTO (SASI_Hs02_00314786), YTHDF1 (SASI_Hs01_00233688), YTHDF2 (SASI_Hs01_00133214), YTHDF3 (SASI_Hs01_00202277), STING (SASI_Hs02_00371843); AllStars negative-control siRNA was purchased from Qiagen.

Real-time PCR. Total RNA was extracted using TRIzol (Invitrogen) according to manufacturer's instructions. cDNA was prepared using qScript XLT cDNA SuperMix (Quantabio 84358). Quantitative real-time PCR (qRT-PCR) was performed using SsoAdvanced Universal SYBR Green Supermix (Bio-Rad 172-5274) in a Bio-Rad C1000 Touch Thermal Cycler with the following primers (Table 3):

TABLE 3

| mRNA | Forward | Reverse |
|---|---|---|
| IFNB1 | 5′-GAAAGAAGATTT | 5′-CCTTCAGGTAAT |
| | CACCAGGG-3′ | GCAGAATC-3′ |
| | (SEQ ID NO: 1) | (SEQ ID NO: 2) |
| GAPDH | 5′-TCTTTTGCGTCG | 5′-ACCAGGCGCCCA |
| | CCAGCCGA-3′ | ATACGACC-3′ |
| | (SEQ ID NO: 3) | (SEQ ID NO: 4) |
| DICER1 | 5′-AAATGGGAAATG | 5′-AGTATACCTGTC |
| | TGATCCAG-3′ | TAAGACCAC-3′ |
| | (SEQ ID NO: 5) | (SEQ ID NO: 6) |
| METTL3 | 5′-CGGGTAGATGAA | 5′-GATTTCCTTTGA |
| | ATTATTTGGG-3′ | CACCAACC-3′ |
| | (SEQ ID NO: 7) | (SEQ ID NO: 8) |
| METTL14 | 5′-ACTAGAAATGCA | 5′-GATTTAAGCTCT |
| | ACAGGATG-3′ | GTGTTCCC-3′ |
| | (SEQ ID NO: 9) | (SEQ ID NO: 10) |
| ALKBH5 | 5′-CGGCGAAGGCTA | 5′-CCACCAGCTTTT |
| | CACTTACG-3′ | GGATCACCA-3′ |
| | (SEQ ID NO: 11) | (SEQ ID NO: 12) |
| YTHDF1 | 5′-CCAGAGAACAAA | 5′-TTTGACTGTCCA |
| | AGGACAAG-3′ | GTAAGGTAG-3′ |
| | (SEQ ID NO: 13) | (SEQ ID NO: 14) |
| YTHDF2 | 5′-CCAAGAGGAAGA | 5′-AGTCCTAATTCT |
| | AGAAAGTG-3′ | CTTGAAGGTC-3′ |
| | (SEQ ID NO: 15) | (SEQ ID NO: 16) |
| YTHDF3 | 5′-ATCAGAGTAACA | 5′-CCCAGGTTGACT |
| | GCTATCCAC-3′ | AAATACAC-3′ |
| | (SEQ ID NO: 17) | (SEQ ID NO: 18) |
| YTHDC1 | 5′-AAGAGAGCTAGA | 5′-ATGCTTCTTTTC |
| | GGCATATC-3′ | TGAACCTG-3′ |
| | (SEQ ID NO: 19) | (SEQ ID NO: 20) |
| PPIA | 5′-CCCACCGTGTTC | 5′-TCTTTGGGACCT |
| | TTCGACAT-3′ | TGTCTGCAA-3′ |
| | (SEQ ID NO: 21) | (SEQ ID NO: 22) |

Type I interferon (IFN) Bioassay. Type I IFN was quantified using the reporter cell line HEK-Blue™ IFN-α/β (InvivoGen, hkb-ifnab) according to the manufacturer's protocol. Briefly, HEK-Blue IFN-α/β cells in 1804, media were incubated with 20 μL NHDF cell culture supernatant at 37° C. and 5% CO2 for 24 hours. Secreted alkaline phosphatase (SEAP) activity was detected by incubating 20 μL HEK-Blue IFN-α/β supernatant with 180 μL QUANTI-Blue™ (InvivoGen, rep-qbl) alkaline phosphatase substrate at 37° C. and 5% CO2 for 15 min. SEAP activity was quantified by measuring the optical density at 640 nm in a SpectraMax M3 plate reader and converted to IFN units using a standard curve generated by quantifying SEAP activity in the supernatant of HEK-Blue IFN-α/β cells incubated with recombinant IFNβ protein (PBL Assay Science, 11415-1).

Indirect Immunofluorescence. NHDFs grown on coverslips were fixed with 4% formaldehyde for 15 min, permeabilized with 0.5% TritonX-100 in PBS for 10 min and then blocked with 4% FBS. Immunostaining was performed using the appropriate primary and secondary antibodies. DNA was stained with 4′,6′-diamidino-2-phenylindole (DAPI). Images were captured using a Leica DM5000 microscope equipped with Leica Imaging Software.

Analysis of m⁶A RNA sequencing. Following demultiplexing, sequence reads were deduplicated using BBtools (bbduk.sh) after which sequencing adapters were removed and low quality ends trimmed using TrimGalore (://github.com/FelixKrueger/TrimGalore) [trim_galore --length 25 infile --clip_R1 5]. Sequence reads were subsequently mapped to hg19 using bowtie2 (Langmead & Salzberg, 2012) and parsed using SAMTools (Li et al., 20019) to yield sorted, indexed BAM files. m⁶A-peak regions were subsequently identified using exomePeak (Meng et al., 2014) and visualized using IGV (Robinson et al., 2011) and GVIZ (Hahne & Ivanek, 2016).

### SUPPLEMENTARY REFERENCES

1. Hahne F, Ivanek R. 2016. Visualizing Genomic Data Using Gviz and Bioconductor. Methods Mol Biol. 1418: 335-351.
2. Langmead B, Salzberg S L. 2012. Fast gapped-read alignment with Bowtie 2. Nat Methods. 9: 357-359.
3. Li H, Handsaker B, Wysoker A, Fennell T, Ruan J, Homer N, Marth G, Abecasis G, Durbin R; 1000 Genome Project Data Processing Subgroup. 2009. The Sequence Alignment/Map format and SAMtools. Bioinformatics. 25: 2078-2079.
4. Liu J, Yue Y, Han D, Wang X, Fu Y, Zhang L, Jia G, Yu M, Lu Z, Deng X, Dai Q, Chen W, He C. (2014). A METTL3-METTL14 complex mediates mammalian nuclear RNA N6-adenosine methylation. Nat. Chem. Biol. 10: 93-95.
5. Meng J, Cui X, Rao M K, Chen Y, Huang Y. 2013. Exome-based analysis for RNA epigenome sequencing data. Bioinformatics. 29:1565-1567.
6. Meng J, Lu Z, Liu H, Zhang L, Zhang S, Chen Y, Rao M K, Huang Y. 2014. A protocol for RNA methylation differential analysis with MeRIP-Seq data and exomePeak R/Bioconductor package. Methods. 69: 274-281.
7. Robinson J T, Thorvaldsdóttir H, Winckler W, Guttman M, Lander E S, Getz G Mesirov J P. 2011, Integrative genomics viewer. Nat Biotechnol; 29(1):24-6.

The foregoing description and examples are intended to illustrate, but not limit the invention.

1. A method of modulating the type I interferon (IFN) pathway in an individual, the method comprising administering to an individual in need thereof one or more agents that inhibit expression or function of METTL3, or METTL14, or ALKBH5, thereby modulating the type I IFN pathway.

2. The method of claim 1, comprising administering to the individual an agent that inhibits the expression and/or function of the ALKBH5, wherein administering the agent results in decreased interferon beta (IFNβ) cytokine production and/or decreased IFNB1 mRNA in cells of the individual, relative to a control IFNβ cytokine production or IFNB1 mRNA value, respectively, from cells to which the agent has not been administered.

3. The method of claim 2, wherein the individual has been diagnosed with an autoimmune disease or inflammatory disease, and wherein the severity of the autoimmune disease or the inflammatory disease is reduced.

4. The method of claim 3, wherein the individual has been diagnosed with the autoimmune disease.

5. The method of claim 4, wherein the autoimmune diseases is positively correlated with the presence of cytoplasmic doubled stranded (dsDNA) in cells of the individual.

6. The method of claim 3, wherein the individual has been diagnosed with the inflammatory disease.

7. The method of claim 6, wherein the inflammatory disease is correlated with the presence of cytoplasmic dsDNA.

8. The method of claim 1, comprising administering to the individual one or more agents that inhibit the expression or function of METTL3 or METTL14, wherein administering the one or more agents results in increased IFNβ cytokine production and/or increased IFNB1 mRNA in cells of the individual, relative to a control IFNβ cytokine production or IFNB1 mRNA value, respectively, from cells to which the agent has not been administered, thereby modulating the type I IFN pathway.

9. The method of claim 8, wherein the individual has been diagnosed with or is suspected of having cancer, or an infection caused by an intracellular parasite, and wherein the severity of the cancer or the infection is reduced.

10. The method of claim 9, wherein the cancer, or the infection caused by the intracellular parasite, is correlated with the presence of cytoplasmic doubled stranded (dsDNA) in cells of the individual.

11. The method of claim 10, wherein the individual has the infection, and wherein the infection is by a DNA virus.

12. The method of claim 11, wherein the dsDNA from the DNA virus is present in cytoplasm of cells of the individual.

13. The method of claim 10, wherein the individual has been diagnosed with the cancer.

14. The method of claim 13, wherein the cancer comprises cancer cells that have cytoplasmic dsDNA.

15. A method for identifying whether or not a test agent affects the function of METTL3/14 and/or ALKBH5, the method comprising:
i) providing a plurality of eukaryotic cells that can express the IFNB1 gene and produce the cytokine interferon beta (IFNβ);
ii) introducing the test agent into the cells; and
iii) determining whether or not there is a change in the amount of IFNβ cytokine production and/or IFNB1 mRNA in the cells subsequent to ii), wherein detecting the change indicates the test agent may inhibit the function of METTL3/14 and/or ALKBH5.

16. The method of claim 15, wherein a decrease in the amount of IFNβ cytokine production and/or I IFNB1 mRNA indicates the agent may inhibit the function of the ALKBH5.

17. The method of claim 16, further comprising testing the agent in control cells with inhibited or reduced ALKBH5 expression, wherein a lack of a decrease in the amount of IFNβ cytokine production and/or IFNB1 mRNA by the control cells indicates the agent inhibits the function of the ALKBH5.

18. The method of claim 16, wherein an increase in the amount of IFNβ cytokine production and/or IFNB1 mRNA indicates the agent may inhibit the function of the METTL3/14.

19. The method of claim 18, further comprising testing the agent in control cells with inhibited or reduced METTL3/14 expression, wherein a lack of an increase in the amount of IFNβ cytokine production and/or IFNB1 mRNA by the control cells indicates the agent inhibits the function of the METTL3/14.

20. The method of claim 15, wherein the plurality of eukaryotic cells comprise cytoplasmic dsDNA.
